(19) United States
(12) Patent Application Publication
(10) Pub. No.: US 2009/0247902 A1
(43) Pub. Date: Oct. 1, 2009

(54) METHOD AND APPARATUS FOR TRANSPORTING A PATIENT SAMPLE BETWEEN A STERILE AND NON-STERILE AREA

(76) Inventors: Julie A. Reichert, Aurora, CO (US); Wayne Siebrecht, Golden, CO (US); Nicholas A. Nelson, San Francisco, CA (US); Laurence R. Shea, San Francisco, CA (US)

Correspondence Address: MORRISON & FOERSTER LLP, 755 PAGE MILL RD, PALO ALTO, CA, 94304-1018, US

(21) Appl. No.: 12/057,245

(22) Filed: Mar. 27, 2008

(51) Int. Cl. A61B 5/00; C12M 1/34

(52) U.S. Cl. 600/573; 435/288.4

(57) ABSTRACT

Devices and methods for performing automated fluid sampling of a patient are disclosed, comprising a patient sampling assembly, test substrates, and a sample transfer assembly to transfer fluid samples from the sampling assembly to the test substrates. The sample transfer assembly may be configured to maintain the sterility of the patient sampling assembly while transferring samples to non-sterile components, test substrates.

### FIELD OF THE INVENTION

The embodiments herein relate generally to sample fluid transfer, including wells, funnels, apertures or depressions for transferring fluid samples from a sterile dispensing area to a non-sterile testing or measurement area.

### BACKGROUND

In the health-care industry, diagnostic testing of physiological or biological samples, such as blood, is a routine, and often cumbersome, task, with physicians requiring a wide variety of specialized tests on patients' samples to support their diagnoses. With in-patient and critical care settings, the frequency of blood sampling places additional demands on hospital staff.

To satisfy this ever increasing demand for analytical data from samples, sophisticated chemical analyzers have been developed over the past 20 years to perform a multiplicity of physical and chemical tests on specially prepared patients' samples. Sample volume requirements have also been reduced substantially, to 100 μL or less for some tests.

### BRIEF SUMMARY

Devices and methods for performing automated fluid sampling of a patient are disclosed, comprising a patient sampling assembly, test substrates, and a sample transfer assembly to transfer fluid samples from the sampling assembly to the test substrates. The sample transfer assembly may be configured to maintain the sterility of the patient sampling assembly while transferring samples to non-sterile components, test substrates.

Some of the embodiments of sample transfer systems may be used to serially analyze body specimens, such as blood or urine, provided by a specimen dispenser. Regardless of the specific testing performed, there is a potential risk of specimen contamination at the testing or the dispensing sites which may affect the proper functioning of the analytical system. The contamination may also affect subsequent specimens or other components of the analytical system. Also, because the transfer of a fluid sample from one component to another component of the analytical system involves the interaction of sterile components and surfaces with non-sterile components and surfaces, not only is there a risk of cross-contamination of sample fluids, but contact with non-sterile components may eventually lead to the spread of infection back to the patient, which may lead to bacteremia, sepsis or even septic shock. Thus, in specific embodiments where the specimen dispenser is attached to a patient for prolonged periods of time, there is a significant risk that infections may spread back into the patient.

Accordingly, some of the embodiments described herein are directed to a sterile transfer shuttle for transferring fluid samples from a sterile dispensing area to a non-sterile testing area while reducing the possibility of cross-contamination between gathered samples and/or preventing contamination of the sterile dispensing area.

For example, vascular access to an artery or vein of a patient may be connected to an analytical system comprising a fluid sample dispenser and a plurality of test substrates. The analytical system further comprises a sample transfer system that is configured to receive a fluid sample from the dispenser using a transfer well. After delivery of a fluid sample from the dispenser, the dispenser and transfer well are separated before the transfer well transfers its fluid sample to a test substrate. A new transfer well is then repositioned for the next sample. By providing a new transfer well for each dispensed sample, the risk of residual fluid affecting later test samples is reduced, but the risk of infecting the dispense with a transfer well that has contacted a non-sterile test substrate has also been reduced.

In one embodiment, a module for insertion into a fluid monitoring system is provided, comprising a well support structure comprising a plurality of transfer wells, wherein each transfer well comprises a cavity comprises an inlet opening, a side wall, and an outlet opening smaller in cross-sectional area than the inlet opening, wherein each transfer well is configured to retain a blood sample having a volume of about 1 μL to about 1000 μL and a hematocrit in the range of about 4% to about 85%. In other embodiments, the transfer well is configured to retain a blood sample having a hematocrit in the range of about 10% to about 65%, and/or having a volume of about 1 μL to about 50 μL. In some embodiments, some or all of the transfer well may be configured to form positive meniscus at the outlet opening using the blood sample. The well support structure may be a disc structure with the plurality of transfer wells arranged in a repeating pattern, which sometimes is a circular repeating pattern. The module may further comprise a housing, in which the well support structure is contained. The housing may comprise a fluid dispenser access opening and a hub interface. In some instances, well support structure may be coupled to the hub interface. The hub structure may be a rotatable hub structure. The housing may further comprise a sterilized compartment. In some embodiments, each transfer well may be further configured to move from a location within the sterilized compartment to a location outside the sterilized compartment. The module may further comprise an absorbent structure in at least one transfer well. The absorbent structure may extend out of the inlet opening the transfer well in some embodiments. The outlet opening of the transfer well may be located in the side wall, or a wall opposite of the inlet opening. In one embodiment, the outlet opening comprises a first transverse dimension that is greater than a second transverse dimension that is perpendicular to the first transverse dimension. In some embodiments, a separation distance between the inlet opening and the outlet opening is less than about 25% of a transverse dimension of the inlet opening, and sometimes less than about 15% of the transverse dimension of the inlet opening. In some embodiments, the outlet opening comprises a cross-sectional area that is at least about 75% of a cross-sectional area of the inlet opening. The cavity may further comprise at least one capillary channel located on the side wall. At least one capillary channel may comprise a radial orientation between the inlet opening and the outlet opening of the cavity. Sometimes, at least one capillary channel comprises an increased width along a direction from the inlet opening to the outlet opening of the cavity.

In another embodiment, a method for performing fluid monitoring is provided, comprising dispensing a fluid sample from a fluid dispenser in fluid communication with a patient, receiving the fluid sample through an inlet opening of a transfer well, tapering the fluid sample from the inlet opening of the transfer well to an outlet opening of the transfer well, forming a positive meniscus of the fluid sample at an outlet opening of the transfer well, and interacting the positive meniscus of the fluid sample with a test medium. The transfer well may be a sterilized transfer well, while in some embodiments, the test medium may not be sterilized. The method may further comprise wiping the fluid dispenser with a cleaning material before and/or after dispensing the fluid sample. The method may further comprise assessing a change in the test medium to determine a characteristic of the fluid sample and may further comprise advancing a new transfer well toward the fluid dispenser. In some embodiments, wiping the fluid dispenser occurs while advancing the new transfer well toward the fluid dispenser. The method may further comprise moving a new test medium while advancing the new transfer well.

In one embodiment, a system for manipulating a fluid sample is provided, comprising a fluid access device configured to be secured to a patient, a fluid sample dispenser, a plurality of fluid test media, a plurality of wells comprising an inlet opening and an outlet opening, wherein the inlet opening is configured to accept a fluid sample from the fluid sample dispenser and the wherein the outlet opening is configured to deliver the fluid sample to a fluid test medium and wherein the outlet opening has a smaller cross-sectional area than the inlet opening. The system may further comprise a fluid pump, at least one fluid source, and/or at least two fluid sources and a distribution valve configured to selectively communicate with at least two fluid sources.

In another embodiment, a system for manipulating a fluid sample is provided, comprising a fluid pathway between a patient and a test medium, a sterilized fluid dispenser located within the fluid pathway, at least one sterilized tapered transfer well within the fluid pathway, at least one bridgeable gap within the fluid pathway between the sterilized fluid dispenser and at least one sterilized transfer well, and a plurality of single-use test substrates. The plurality of single-use test substrates may comprise irreversible reaction reagents.

In some embodiments, a system for manipulating a fluid sample is provided, comprising a fluid access device configured to be secured to a patient, a fluid sample dispenser, a plurality of test substrates, and a plurality of retaining structures comprising an opening, wherein the opening is configured to accept a fluid sample from the fluid sample dispenser. The system may further comprise a test substrate advancement mechanism configured to insert at least one of the plurality of test media into at least one of the retaining structures. The plurality of retaining structures may further comprise an insertion opening, and wherein the test media advancement mechanism is configured to insert at least one of the plurality of test media into the insertion opening. In some embodiments, the retaining structure may be an absorbent structure. In some further embodiments, the retaining structure is selected from a group consisting of a foam, fibrous structure and a woven structure. The retaining structure may further comprise a well cavity in which the absorbent structure is located. The plurality of test substrates may comprise a plurality of irreversible reaction reagents. The system may optionally further comprise a compression structure, and the compression structure may be configured to press a test substrate of the plurality of test substrates against a retaining structure of the plurality of retaining structures.

In another embodiment, a method for performing fluid monitoring is provided, comprising dispensing a fluid sample from a fluid dispenser in fluid communication with a patient, receiving the fluid sample through an opening of a retaining structure, contacting a test substrate to the retaining structure, transferring at least a portion of the fluid sample from the retaining structure to the test substrate, positioning the test substrate at an analysis site, analyzing the test substrate with a sensor assembly at an analysis site, and removing the test substrate from the analysis site. The method may further comprise absorbing the fluid sample with the retaining structure, irreversibly reacting the fluid sample with the test substrate, and/or inserting the test substrate through the opening of the retaining structure. Inserting the test substrate through the opening of the retaining structure may be performed before transferring at least a portion of the fluid sample from the retaining structure to the test substrate. In some embodiments, the method may further comprise inserting the test substrate through an insertion opening of the retaining structure, wherein the insertion opening is different than the opening of a retaining structure that receives the fluid sample. The method may further comprise compressing the retaining structure using the test substrate and/or applying force against the test substrate using a compression member. Removing the test substrate from the analysis site may comprise pivoting the test substrate away from the analysis site.

### BRIEF DESCRIPTION OF THE DRAWINGS

These and other features and advantages will be appreciated, as they become better understood by reference to the following detailed description when considered in connection with the accompanying drawings, wherein:

FIG. 1 is a schematic diagram of fluid-sample handling system;

FIG. 2 is a cross-sectional view of one embodiment of a transfer well;

FIGS. 3A through 3C depict cross-sectional views of the transfer well in FIG. 2 being filled with a fluid sample;

FIG. 4 is a cross-sectional view of another embodiment of a transfer well;

FIGS. 5A through 5C depict cross-sectional views of the transfer well in FIG. 4 being filled with a fluid sample;

FIG. 6 is a cross-sectional view of another embodiment of a transfer well;

FIGS. 7A through 7C depict cross-sectional views of the transfer well in FIG. 6 being filled with a fluid sample;

FIGS. 8A through 8D depict a variety of test strip configurations;

FIGS. 9A and 9B are perspective views of another embodiment of a transfer structure;

FIG. 10A depicts the functional elements of an embodiment of an automated fluid access device integrated with a sample dispensing device; FIG. 10B depicts an exemplary embodiment of the blood sample dispensing device;

FIG. 11 is a schematic cross-sectional view of an embodiment of a transfer well;

FIG. 12 is another schematic cross-sectional view of a transfer well with a fluid sample;

FIG. 13 is a schematic cross-sectional view of the transfer well of FIG. 12 with another fluid sample;

FIG. 14 is a schematic cross-sectional view of another embodiment of a transfer well;

FIG. 15 is a schematic cross-sectional view of another embodiment of a transfer well;

FIG. 16 is a schematic cross-sectional view of another embodiment of a transfer well;

FIG. 17A is a superior view of one embodiment of a transfer well; FIG. 17B is a superior view of another embodiment of a transfer well;

FIG. 18 is a schematic cross-sectional view of a transfer well groove;

FIG. 19 is a schematic cross-sectional view of a transfer well projection;

FIG. 20A is a component view of one embodiment of a transfer well cartridge; FIG. 20B is a perspective view of an assembled and unused transfer well cartridge of FIG. 20A; FIG. 20C is a perspective view of the transfer well cartridge in FIG. 20B after use;

FIG. 21A is a schematic cross-sectional view of the transfer well cartridge in FIG. 20B; FIG. 21B is a schematic cross-sectional view of the transfer well cartridge in FIG. 20C;

FIG. 22A is a component view of an embodiment of a transfer well cartridge with test medium component; FIG. 22B is a schematic cross-sectional view of the transfer well cartridge of FIG. 22A;

FIG. 23 is another embodiment of a test medium component;

FIG. 24 is another embodiment of a test medium component;

FIG. 25 is a schematic illustration of a transfer well cartridge and a test medium disc;

FIG. 26 is a schematic cross-sectional view of another embodiment of a transfer well;

FIG. 27 is a schematic cross-sectional view of another embodiment of a transfer well;

FIG. 28 is a schematic cross-sectional view of another embodiment of a transfer well;

FIG. 29A is a component view of another embodiment of a transfer well cartridge; FIG. 29B is a schematic cross-sectional view of the transfer well cartridge in FIG. 29A;

FIG. 30 is a perspective view of another embodiment of a test medium component;

FIG. 31A is a component view of another embodiment of a transfer well cartridge; FIG. 31B is a detailed view the transfer well cartridge of FIG. 31A; and

FIGS. 32A and 32B are schematic cross-sectional views of another embodiment of a transfer well, comprising an absorbent material, before and during compressive fluid transfer, respectively;

FIGS. 33A and 33B are schematic cross-sectional views of another embodiment of a transfer well, comprising an absorbent material, before and during compressive fluid transfer, respectively;

FIGS. 34A and 34B are schematic cross-sectional views of an embodiment of a transfer structure comprising an absorbent material before and during compressive fluid transfer, respectively;

FIGS. 35A and 35B are schematic cross-sectional views of an embodiment of a transfer structure comprising an absorbent material before and during compressive fluid transfer, respectively; and

FIGS. 36A and 36B are schematic cross-sectional views of an embodiment of a transfer structure comprising a transfer ring before and after droplet capture, respectively; FIG. 36C is a superior elevational view of the transfer structure in FIGS. 36A and 36B.

### DETAILED DESCRIPTION

“Sample”, “fluid sample”, or “sample fluid” are used interchangeably in the present specification. Sample, as used herein, includes any biological or physiological sample from blood, semen, plasma, urine, sweat, or fluid derived from the body or tissue of a patient. The sample may be a direct sample from the patient, or be patient sample which is extracted, diluted, suspended, or otherwise treated, in solution. Other types of samples, including antibiotic dosages or other fluidized materials are also included.

In some embodiments, a sterile transfer medium is provided for transferring a physiological sample from a sterile dispensing area to a non sterile testing or measurement area. The transfer medium may be a support structure for receiving samples of fluid. The support structure may comprise a plurality of micro depressions, micro apertures, micro funnels, or micro wells, generally and collectively referred to as “transfer wells”, arranged on the support structure. The arrangement of transfer wells may include any of a variety of layouts, including but not limited to radial, linear, and circumferential layouts. These transfer wells may be used to capture, temporarily store, and/or enable the transfer of fluid samples. In some embodiments, the support structure may be a generally planar or plate-like structure, such as a disc, with transfer wells organized along at least one radius of the disc with adjacent transfer wells equidistant from one another. In other embodiments, the receiving structure may have one or more curves or angles, such as a dome or a plate with angled sidewalls.

In one specific embodiment, the transfer well comprises a generally funnel-type shape with an upper end configured to receive a fluid sample and a lower end configured to transfer the fluid sample to another structure. The upper end of the transfer well may comprise a diameter that is larger than the lower end and tapers into the lower, narrower diameter end. A drop or volume of solution to be analyzed is dispensed to and held by the transfer well. In this particular embodiment, the transfer well is configured with a geometry that balances surface-tension and volume extremes of the solution to be analyzed. The transfer well is then manipulated in time and/or space to interact with a test medium that is brought into contact with the lower, narrower diameter end of the transfer well, or the fluid sample retained by the transfer well. The contact or other interaction causes the fluid sample in the transfer well to be transferred to the test medium. In some embodiments, the duration of contact may be fixed or pre-determined, but in other embodiments, the duration of physical contact may be based upon real-time sensor feedback. The test medium may be configured a strip, a bead, or a well, for example, and may comprise a gel, a foam, or a powder, for example.

In one particular embodiment, the test medium comprises an absorption structure or material. In some embodiments, the absorptive properties of the absorption structure may reduce variations relating to sample fluid volume transfer. For example, the absorption structure may reduce the degree of precision used to align the dispense valve to a transfer well, and/or reduce any variations relating to gravity, surface tension/surface texture, or the duration and/or angle of contact. In some embodiments, the absorption structure may further comprise one or more other substances that interact with the fluid sample to modify its analytical characteristics and/or to initiate any reactions used to measure a fluid parameter. In some embodiments, analytical measurements may be performed directly on the absorption structure, but in other embodiments, the absorption structure may be compressed or manipulated to transfer the fluid sample from the absorption structure to another location for analysis.

According to another embodiment, the transfer well comprises a lower end with an angled bottom so that a sample fluid is transferred to a test medium from one side of the lower end of the transfer well. In some embodiments, the transfer well may comprise a soft, pliable bottom.

In some embodiments, the transfer wells are sterilized or sterilizable. Prior to use, the transfer wells are kept in a sterile environment or compartment. After being subjected to a fluid sample, a transfer well is transferred from the sterile environment or compartment. Thus, used transfer wells may be physically separated from the unused transfer wells, which are maintained in the sterile environment. Once all of the transfer wells of the sample receiving structure move out of the sterile area or are used up, the receiving structure is disposed. Separating fluid dispensing and use from the sterile environment may resist or prevent contamination of the unused, sterile wells.

In another embodiment, the receiving structure is a closed-bottomed well or depression, referred to as a soak well, integrated with a base support. The base support comprises a cap that incorporates an opening. The cap covers at least a portion of the soak well surface. The soak well remains exposed through the opening, thereby allowing sample fluid stored in the soak well to be extracted, through wicking or capillary action, via the opening in the cap. A test medium, including but not limited to electrochemical test strips, is provided with a capillary port at one end. The capillary port end of the test strip is brought in contact with the cap opening, permitting analyte detection and/or measurement of the sample fluid contained in the soak well. In some embodiments, the soak wells are single use, and a plurality of soak wells may be organized in any of a variety of configurations, including but not limited to linear arrays, circular or arcuate arrays, spiral or helical arrangements, two-dimensional matrices, etc.

In other embodiments, the receiving structure comprises wells having a single opening configured to receive a fluid sample from a sample dispenser. After dispensing a fluid sample into a well, a test substrate is then placed in fluid communication with the well to initiate a test reaction. In some embodiments, the test substrate may be directed inserted into the well, but in other embodiments, a tubular or grooved fluid transport structure is inserted through the single opening to transport the fluid sample to the test substrate. In some embodiments, the test reaction is analyzed while the test substrate remains in fluid communication with the well, while in other embodiments, the test substrate or fluid transport structure is removed from the well prior to analysis.

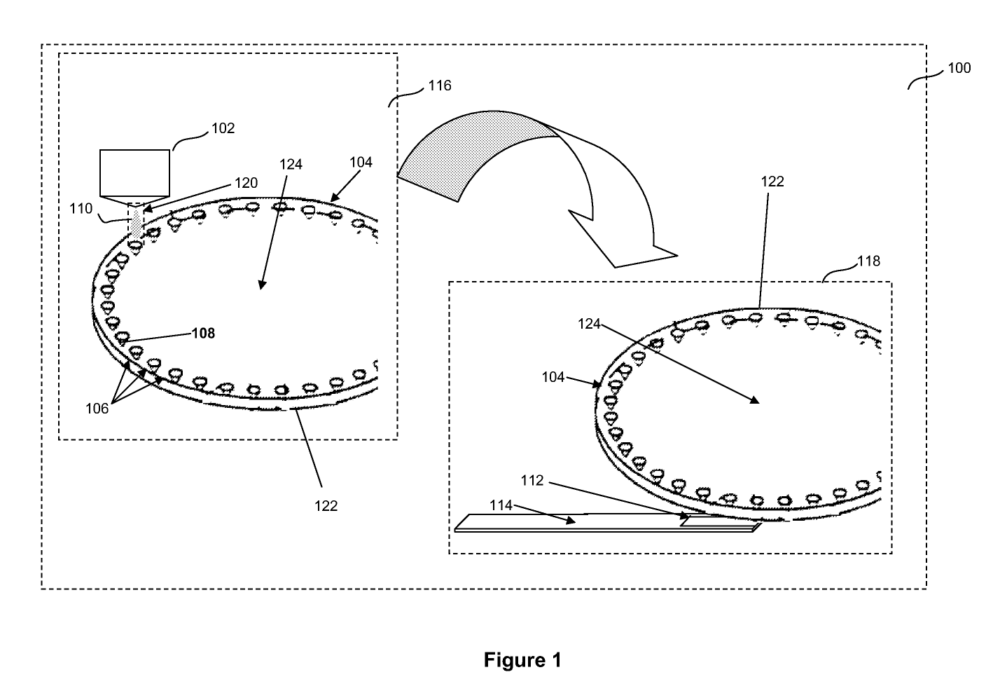

FIG. 1 is a schematic diagram of one embodiment of a fluid dispensing and transfer system 100, comprising a fluid-sample dispensing structure 102 with a transfer structure 104 that comprises a plurality of sample transfer sites 106. In some embodiments, sample transfer sites 106 may comprise transfer wells 108, which are spaces or sites where fluid samples 110 can be temporarily stored and then dispensed from the transfer structure 104 to a test medium 112. Test medium 112 may be supported on a test medium support structure 114, which may contain more a plurality of test mediums 112.

During the dispensing phase, dispensing structure 102 dispenses fluid sample 110 to transfer structure 104, while in the transfer phase, transfer structure 104 transfers fluid sample 110 to test medium 112. The dispensing structure 102 may have a fixed position in the dispensing zone 120, or the dispensing structure 102 may be moved in and then out of the dispensing zone 120 for the dispensing phase. The spacing of the dispensing structure 102 and the transfer structure 104 during the dispensing phase 116 may be generally fixed, or may be variable. In some embodiments, variable spacing may be used to facilitate transfer of the fluid sample and/or to control the volume of the fluid sample.

In some embodiments, the variable spacing may pre-programmed or controlled in real time based one or more factors. For example, in some embodiments, the spacing may be altered depending upon the size of the fluid sample 110 used for a particular test, or based upon the viscosity or other features of the fluid sample 110, e.g. the hematocrit of a blood sample may be used as a measure for blood viscosity. In embodiments where the spacing is variable, the dispensing structure 102 or the transfer structure 104, or both, may be moved to provide the variable spacing. In some embodiments, the spacing between the dispensing structure 102 and the transfer structure 104 during the dispensing phase 116 may be vary depending upon the size of the fluid sample and/or the particular contents of the fluid sample. In a blood monitoring system, for example, the spacing may vary based upon the size of the blood drop used for a particular test substrate, as well as the hematocrit and/or glucose level of the blood sample. In some embodiments, the hematocrit and/or glucose level may affect the viscosity and/or surface tension of the blood drop and its interactions with the sample dispenser and target structure.

In some embodiments where contact between the dispensing structure 102 and the transfer structure 104 occurs during the dispensing phase, either structure or both structures may be moved. For example, in some embodiments, the dispensing structure 102 may be displaced toward the transfer well 108, while in other embodiments, the transfer well 108 may be displaced toward the dispensing structure 102.

In other embodiments, dispensing structure 102 and transfer structure 104 do not contact each other during the dispensing phase. In some embodiments, the spacing between dispensing structure 102 and transfer structure 104 may be configured so that during the transfer of a fluid sample 110, the fluid sample 110 is in contact with both dispensing structure 102 and transfer well 108. In other embodiments, fluid sample 110 may separate from dispensing structure 102 and may have a transient mid-air position before contacting transfer structure 104 or the sample dispensing site 106 of transfer structure 104.

The duration of the dispensing phase may be fixed, pre-determined or variable based upon sensor feedback. The dispensing phase may be further characterized by one or more subphases, which may be exclusive in some embodiments and overlapping in other embodiments. In some embodiments, the dispensing phase may include a fluid sample formation phase, during which a volume of fluid is generated by the dispensing structure 102. The dispensing phase may also include a sample movement phase, where there is a net shift in the volume of the fluid sample 110 into the sample transfer site 106.

The transfer phase may also involve movement and timing of transfer structure 104 and/or support structure 114 of test medium 112. In some embodiments, either transfer structure 104 and/or support structure 114 may be moved to achieve transfer of fluid sample 110. In some embodiments, the transfer of fluid sample 110 from transfer well 108 to test medium 112 is occurring generally at the same location, while in other embodiments, transfer structure 104 may be rotated or moved to a different location before transferring fluid sample 110 to test medium 112. Movement of transfer well 108 to a different location may be beneficial where the transfer of the fluid sample 110 to the test medium 112 may potentially splatter or otherwise contaminate or affect the dispensing structure 102 with the fluid sample 110. The movement of transfer well 108 may comprise a translational movement, rotational movement, pivoting movement, or combination thereof. In some instances transfer site 106 or transfer well 108 may separate from transfer structure 104 before, during or after use.

After the testing of fluid sample 110 is completed, one or more optional procedures may be performed to maintain the functioning of fluid dispensing and transfer system 100. For example, dispensing structure 102 may be cleaned by a cleaning assembly to reduce the risk of clogging, or the risk that residuals from prior fluid samples may affect subsequent fluid samples. The cleaning assembly may include but is not limited to mechanical wiping and/or absorbent structures, as well as flushing dispensing structure 102 with fluid. In some embodiments, between sampling procedures, fluid dispensing and transfer system 100 may periodically perform a calibration test whereby one or more calibration procedures are performed on the system 100 as a whole or one or more components of the system 100. In some embodiments, the calibration procedure may include but is not limited to the analysis of one or more references solutions.

In some embodiments, system 100 may be configured to so that transfer structure 104 may be moved independent of support structure 114. Independent movement permits changing sample transfer sites 106 with each sample delivered, as well as use of transfer site 106 to transfer multiple fluid samples 106 to different test mediums 112. In other embodiments, transfer structure 104 and support structure 114 are configured to move synchronously.

The change in transfer sites 106 may occur just before the next testing procedure or just after the prior testing procedure. In some embodiments, between testing procedures the transfer structure 104 may be removed from the dispensing zone 120, or a portion of the transfer structure 104 without a transfer site 106 is positioned in the dispensing zone 120.

In some embodiments, the same transfer site 106 may be used multiple times within a particular time frame. This time frame may be about 0.4 seconds to about 5 minutes, sometimes about 1 second to about 2 minutes, and other times about 1 second to about 40 seconds. In some embodiments, use of a transfer site 106 with a particular test medium 112 may result in chemical or other type of contamination of the transfer site 106, so that a new transfer site 106 is programmed for use with the next test.

Referring still to FIG. 1, in some embodiments, transfer structure 104 comprises a circular transfer disc 122 comprising a plurality of transfer wells 108 formed around the circumference of the disc 122. Although this particular embodiment provides a single circular arrangement of transfer wells 108, in other embodiments, additional circular arrangements of transfer wells 108 may be provide in the interior region 124 of the disc 122. The spacing between transfer wells 108 at other radii of the disc 122 may be uniform or non-uniform. In other embodiments, transfer wells 108 may be arranged in a helical arrangement. In still other embodiments, a linear array or a two-dimensional matrix of transfer wells 108 may be provided on disc 122. Disc 122 in FIG. 1 depicts transfer wells 108 with generally equal distances between adjacent transfer wells 108, but in other embodiments, the spacing may be different. Also, the circumferential spacing between groups of transfer wells 108 located along different radii of the disc may also be the same or different. Also, the radial spacing between groups of transfer wells 108 may be the same or different. The number and spacing of the transfer wells 108 may depend upon the volume of the selected fluid sample and the spacing that reduces the risk of cross-spillage or contamination of adjacent transfer wells filing dispensing fluid samples from the dispensing structure. The configuration of each transfer well 108 may also vary within the same transfer structure 104.

The disc 122 may be made of polystyrene, PET, or any other suitable plastic or other material known to persons of ordinary skill in the art. The disc may comprise a rigid, semi-rigid or flexible material or structure. The disc 122 may be tempered during manufacture with a softening material, so that crystalline rigidity, and resultant tendency to crack or chip, is reduced. For example, the disc 122 may be manufactured out of a blend of polystyrene, approximately 90% or more, along with an additive of butyl rubber to increase the flexible and damage resistance of the disc, for example.

Although the some embodiments described herein are directed to the use of non-sterile test substrates, in other embodiments, sterile substrates may be used. Sterile substrates, however, are sometimes associated with a lower shelf life because the sterilization procedures often involve thermal energy which may degrade or alter the product test characteristics. In some embodiments, the transfer wells 108 are configured as single-use. For example, the transfer wells 108 may be sterilized and become unsterile after use, and/or the transfer wells 108 may retain some residual material from the fluid sample 110 after use.

In some embodiments, the used transfer wells are physically separated from the unused transfer wells by a wall, compartment or film lining, for example. Once all transfer wells 108 on the sample disc 122 have been used once, the disc 122 the disc may be removed and disposed. Since the transfer wells 108 are single-use, they act as a sterilized disposable apparatus used to transport a patient sample between a sterile dispensing area 116 and a non-sterile sample testing measurement area 118.

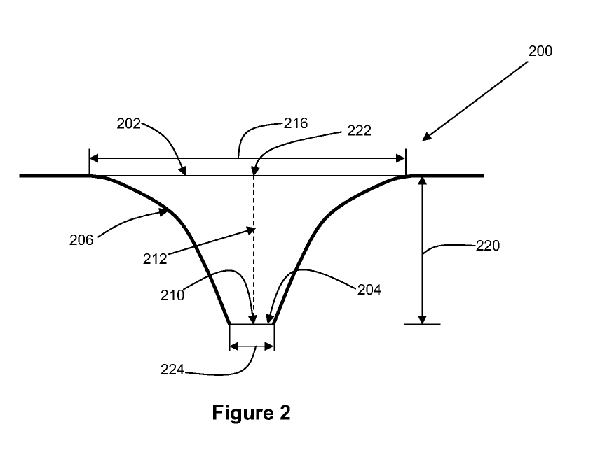

FIG. 2 is a cross-sectional view of one embodiment of a transfer well 200, comprising a configuration with a first opening 202, a second opening 204 that is smaller than the first opening 202, and walls 206 therebetween. In this particular embodiment, the first opening 202 is the inlet opening while the second opening 204 is the outlet opening of transfer well 200. In some embodiments, more than one inlet opening and/or more than one outlet opening may be provided on a transfer well. In the particular embodiment in FIG. 2, the centers 208 and 210 of the first and second openings 202, 204, respectively, are generally aligned along a perpendicular axis to the first opening 212, but in other embodiments, the centers of the openings need not be in alignment. In the embodiment in FIG. 2, the first and second openings 208 and 210 each have a planar configuration that is generally parallel with the planar configuration of the other opening. In other embodiments, however, the openings may not have a planar configuration, and/or may not be parallel. Examples of other transfer well embodiments will be described later below.

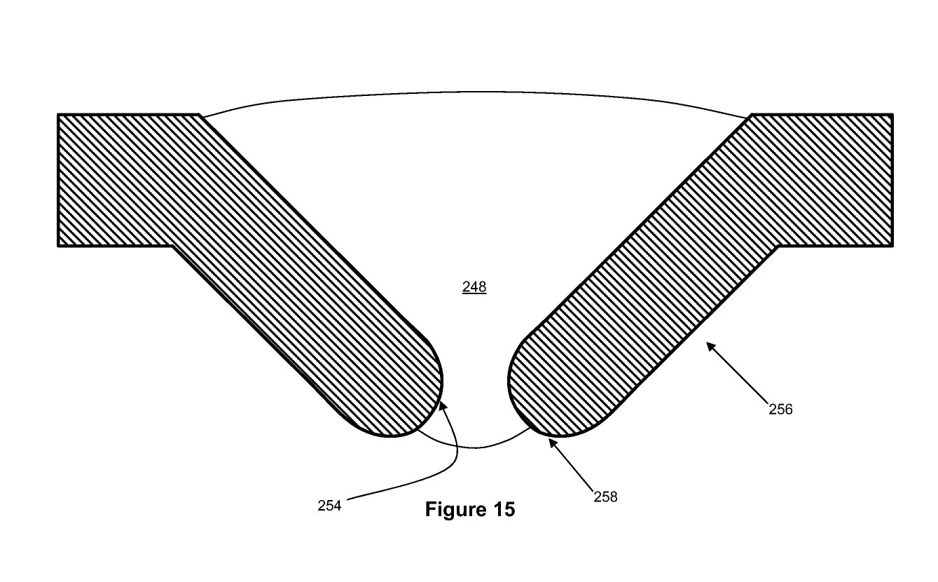

The walls of the transfer well 200 may be linear, angled, or curved. Although the walls in FIG. 2 are depicted as symmetrical tapering walls with respect to a central perpendicular axis 212 to the first opening 202, in other embodiments the walls may be asymmetrical to that axis. A shown in the example in FIG. 2, transfer well 200 may have a generally inverted conical or funnel-type shape. In embodiments with generally tapering walls, the rate of tapering may vary between first opening 202 and second opening 204. In some embodiments, transfer well 200 may have one or more sections where the cross-sectional shape or the dimension of the transfer well (as measured along axis 212) remains generally constant or is even increasing. For example, FIG. 11 depicts one example of a transfer well 214 comprising a frusto-conical portion 216 and a cylindrical portion 218. The cross-sectional shape of a transfer well may be circular, as depicted in FIGS. 1 and 2, but a transfer well may also be elliptical, square, rectangular, triangular, or any other shape. Also, the cross-sectional shape of a single transfer well may be uniform or non-uniform along an axis of the transfer well. The plurality of transfer wells on a disc or other transfer structure may also be uniform or non-uniform in their configurations.

Referring back to FIG. 2, the longitudinal height 220 between first opening 202 and second opening 204 (or the distal most point 221 along a perpendicular axis 212 of first opening 202) of transfer well 200 may be about 1 mm to about 5 mm, sometimes about 2 mm to about 3 mm, and other times about 0.4 mm to about 1.8 mm. This longitudinal height may be the same or different than the longitudinal distance between centers 208 and 210 of the first and second openings 202 and 204, respectively. First opening 202 of the transfer well 200 may have a diameter 222 or transverse dimension of about 2 mm to about 10 mm, sometimes about 4 mm to about 8 mm, and other times about 1 mm to about 6 mm. Second opening 204 of the transfer well 200 may have a diameter 224 or transverse dimension of about 0.3 mm to about 4 mm, sometimes about 1 mm to about 3 mm, and other times about 0.8 mm to about 2 mm. In some embodiments, transfer well 200 may be characterized by the ratio between diameters 222 and 224 or transverse dimensions of first opening 202 and second opening 204, respectively, or the ratios between a diameter 222 or 224 and height 220 of transfer well 200, or ratios involving any other dimension or geometric feature of transfer well 200.

Although the openings, walls and other dimensions of the transfer well 200 may be used to determine the volume of the transfer well 200, the actual volume of fluid sample 110 retained by the transfer well 200 and/or transferred out of the transfer well 200 may be more or less than the calculated volume of the transfer well 200. These factors may include the amount of fluid dispensed by the dispensing structure, the viscosity, density and surface tension of the fluid sample, properties of the transfer well 200 relating to its geometry and its hydrophilic/phobic properties, and the interactions of these properties that determine the contact angle between the fluid sample and the walls of the transfer well 200, for example. In some embodiments, walls 206 of transfer well 200 may be electrically charged to alter its surface interactions with a fluid sample. Based upon the characteristics and the volume of the sample fluid and the geometry of the transfer wells, the shape and volume of the fluid sample as retained by the transfer well may be determined.

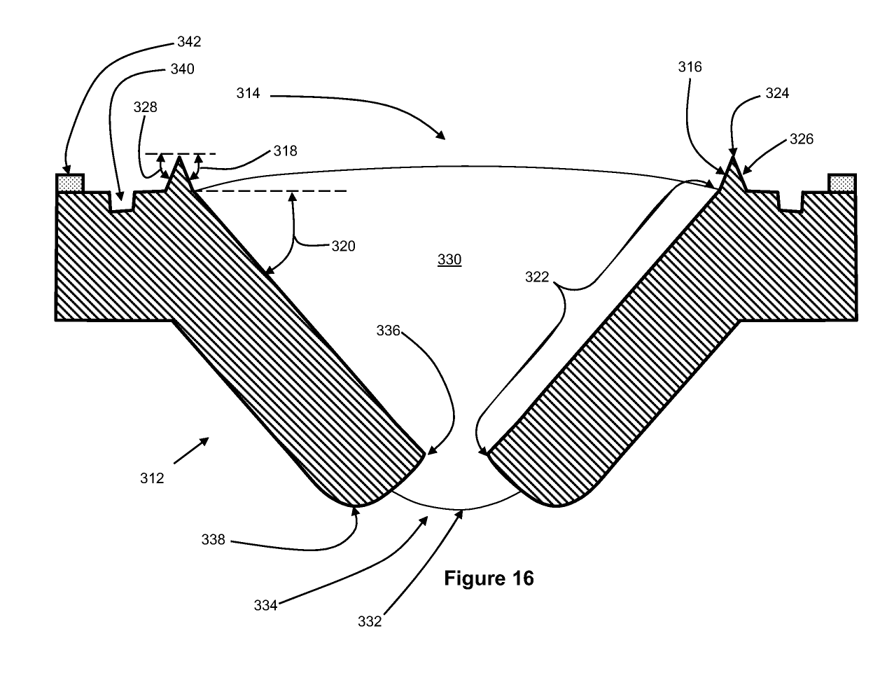

In some embodiments, the fluid sample may form a generally positive meniscus, a generally negative meniscus, or no meniscus about each of first and/or second openings 202 and 204 of transfer well 200. For example, in FIG. 12, the transfer well 226 is configured such that with a particular volume and type of fluid sample 228, a negative meniscus 230 and 232 is formed at both the first and second openings 234 and 236, respectively. In other embodiments, the transfer well may be configured to form a positive meniscus at the one or more opening with a particular fluid sample dispensed by the dispensing structure. In FIG. 13, for example, transfer well 226 from FIG. 12 is filled with a fluid sample 238 comprising a larger volume or a different type of fluid which generates a positive meniscus 230 at A positive meniscus 240 at first opening 234 and a generally flat fluid surface 241 at second opening 236, for example. In some embodiments, providing a fluid sample volume sufficient to generate a positive meniscus at the outlet opening of the transfer well may also generate a positive meniscus at the inlet opening. In other embodiments, a positive meniscus may be formed at both openings 234 and 236, or only at second opening 236. In some embodiments, a positive meniscus at the outlet opening may be beneficial to facilitate transfer of a fluid sample from the transfer well to the test medium without requiring actual contact between the two structures. In some of these embodiments, the contact of the fluid meniscus to the test medium may be sufficient to draw out the fluid sample from the transfer well and onto the test medium.

Some embodiments may include additional features to facilitate or constrain fluid sample delivery to a particular volume or range of volumes, or to achieve a particular morphology of the fluid sample as retained in the transfer well. For example, one or more openings of the transfer well may comprise a protruding edge or a recessed edge. In FIG. 14, for example, a protruding edge 242 is provided at an inlet opening 244 of the transfer well 246 to constrain the dispensed fluid sample 248 from spilling out and contaminating adjacent transfer wells while providing fluid sample 248 with a positive meniscus 250 at the outlet opening 252. In the example in FIG. 15, one or more openings 254 of a transfer well 256 may be provided with a rounded edge 258, in contrast to a squared edge 260 as depicted in FIG. 13. In some embodiments, a rounded edge 258 may increase movement of the fluid sample 248 through the outlet opening 252, increasing the likelihood of forming a positive meniscus 250, or to reduce the amount of the fluid sample 248 left as a residual.

FIG. 16 depicts another embodiment of a transfer well 312. In this particular embodiment, the inlet opening 314 comprises an inner wall 316 with a steep inner inlet angle 318 than the middle angle 320 of the middle portion 322. In some embodiments, the inlet opening 314 may also comprise an edge 324 with an acute angle, comprising an outer wall 326 that has a steep outer inlet angle 328. In some embodiments, the configurations of the inlet opening 314 comprising increased inner and outlet angles 318 and 328 may reduce the risk that a fluid sample 330 may wick over the edge 324. Wicking over the edge 324, in some instances, may increase the amount of the fluid sample 330 retained by the transfer well 312 after transfer to a test medium. The risk of wicking may increase, for example, when a fluid sample 330 with a larger volume is delivered, or when a fluid sample 330 is delivered eccentrically with respect to the inlet opening 314. In some embodiments, an increased inner inlet angle may also redistribute some of the forces acting on the fluid sample 330 from the inner wall 316 to the fluid sample 330, which may increase the chance of forming a positive meniscus 332 at the outlet opening 334 of the transfer well 312. In some embodiments, the inner inlet angle 318 and/or the outer inlet angle 328 lie in the range of about 0 degrees to about 180 degrees, sometimes about 45 degrees to about 90 degrees, and other times about 60 degrees to about 85 degrees. In some embodiments, the edge 324 comprises an angle of about 0 degrees to about 180 degrees, sometimes about 5 degrees to about 90 degrees, and other times about 15 degrees to about 45 degrees.

In the particular embodiment illustrated in FIG. 16, the outlet opening 334 of the transfer well 312 comprises an inner edge 336 and an outer edge 338. Each of the edges 336 and 338 may have any of a variety of configurations, whether rounded or sharp, textured or smooth, for example. In some embodiments, the outlet opening 334 may be configured with an inner edge 336 with a sharp or angled configuration. In some instances, a sharp or angled configuration may facilitate the separation of the fluid sample 330 from the transfer well 312 upon completing the transfer of the fluid sample 330 to a test medium. This may occur by isolating gravitational forces acting on the fluid sample 330 to overcome adhesive forces between the fluid sample 330 and the transfer well 312. In some embodiments, the outer edge 338 of the outlet opening 334 may comprise a round configuration. In some instances, the round configuration may facilitate the formation of a positive meniscus 332 at the outlet opening 334, for example, by taking utilizing the adhesive forces between the fluid sample 330 and the transfer well 312 to draw out the fluid sample 330.

The transfer well 312 may also comprise an optional groove or moat 340 about the inlet opening 334. The moat 340 may be act as a retaining space for any portion of the fluid sample 330 that may have spilled over or been dispensed outside of the inlet opening 334. In some instances, the moat 340 may reduce the risk that portions of the fluid sample 330 may contaminate other transfer wells or other portions of the transfer support structure. The moat 340 may be configured to substantially surround the entire inlet opening 334 or a portion thereof. In some embodiments, multiple moats 340 may be provided. In some embodiments, a helical or variable spacing moat from the inlet opening 334 may be provided. In some embodiments, the helical moat comprises at least one turn around the inlet opening, but in other embodiments, the helical moat may comprises two or three turns or more turns. In embodiments comprising multiple moats, the multiple moats may be arranged serially about the perimeter of the inlet opening, and/or concentrically about the inlet opening. The overall configuration of the moat or moats may be circular, oval, square, rectangular, triangular, or any other polygon or any other open or closed shape. In some embodiments, the moat may comprise a plurality of non-elongate depressions and/or other surface projections or irregularities.

The transfer well 312 may also optionally comprise one or more wiping, cleaning and/or absorbent structures 342 about its inlet opening 314. In some embodiments, the cleaning structures 342 are configured to contact or clean the fluid dispenser (not shown) before and/or after dispensing a fluid sample 330. In some embodiments, these cleaning structures 342 may be used to clean the dispensing device during the touch-off cycle, and/or control any satellite spray. The cleaning structures 342 may also act to retain any other portions of a dispensed fluid sample not contained by the transfer well 312 or the moat 340.

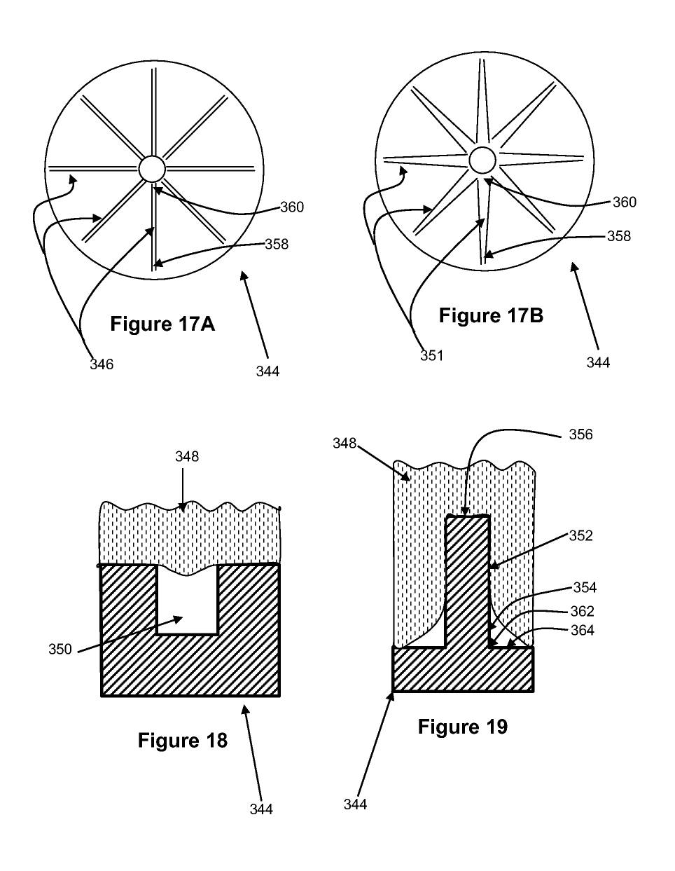

The inner surface of the transfer well may be smooth, or may comprise any of a variety of surface structures or textured features. FIG. 17A depicts one embodiment of a transfer well 344 comprising one or more surface structures 346. Although the surface structures 346 depicted in FIG. 17A are radially oriented and equally spaced, the orientation and the spacing of a surface structure 346 need not be uniform. The cross-sectional shape of the surface structure 346 may be any of a variety of shapes, including but not limited to a square, a rectangle, a circle, an oval, a triangle, a trapezoid, or any other polygons or other shape. The cross-sectional shape, cross-sectional area, height/depth, width, and/or length of the surface structures 346 may vary along one or more dimensions. Other orientations and arrangements may be used, including but not limited to concentric, helical or a random configurations. The use of surface structures 346 is not limited to transfer wells 344 having a circular cross-sectional shape. In some embodiments, the use of surface structure 346 may facilitate the transfer of a fluid sample 348 out of the transfer well 344. This may occur in some instances by reducing the adhesive forces between the fluid sample 344 and the transfer well, and/or by leveraging the gravitational forces acting on the fluid sample 330 to overcome the adhesive forces. Referring to FIGS. 18 and 19, the surface structures 346 may comprise depressions 350 and/or projections 352, respectively. Referring to FIG. 18, a groove 350 in the transfer well 344 may reduce the adhesive forces between the fluid sample 348 and the transfer well 344 by utilizing the cohesive forces of the fluid sample 348 which will resist the filling of the groove 350. In some embodiments, the groove 350 may act as a capillary channel to facilitate movement of the fluid sample 348 along the direction of the groove 350. In some embodiments, the groove 350 have a radial configuration in the transfer well 344 as depicted in FIG. 17A. In some of these embodiments, the groove 350 may have a variable depth and/or width along its longitudinal length between the its outer end 358 and its inner end 360. In one particular embodiment, depicted in FIG. 17B, the width and/or depth of the grooves 351 increases toward their inner ends 360.

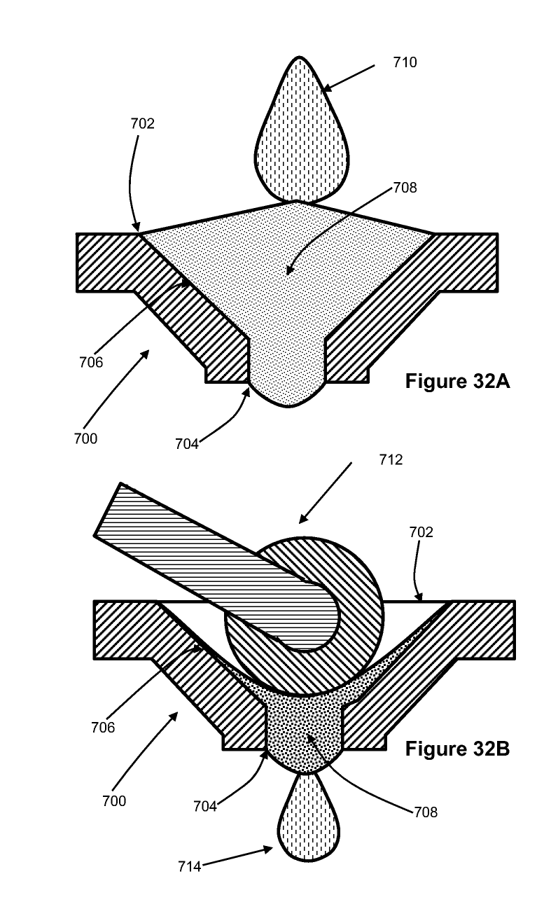

FIG. 32A depicts another embodiment, comprising a transfer well 700 with an inlet opening 702, an outlet opening 704, and a well cavity 706 therebetween. The well cavity 706 comprises a sponge or absorbent element 708 that can releasably retain the fluid sample 710 dispensed to the transfer well 700. In FIG. 32B, when the absorbent element 708 is compressed or squeezed with a compression member 712, a fluid sample 714 is transferred out of the transfer well 700 through the outlet opening 704. Although the compression element 712 is shown in FIG. 32B as acting on the absorbent element 708 through the inlet opening 702, in other embodiments, the compression element 712 may be used through the outlet opening 704 or another opening provided on the transfer well. In some embodiments, the absorbent element may be compressed by a test substrate or test strip, with or without supplemental use of a compression member either acting on directly on the absorbent structure or indirectly through the test substrate or test strip. In still other embodiments, the absorbent material 708 may transfer a fluid sample by contact and does not require compression by a compression member. The compression member 712 depicted in FIG. 32A is a roller mechanism, but in other embodiments, the compression member 712 may comprising a tamping member, push rod or other type of compression member.

As noted in between FIGS. 32A and 32B, the fluid sample 710 dispensed to the transfer well 700 need not have the same volume as the fluid sample 714 transferred out of the transfer well 700. The absorbent element 708 may be beneficial in some embodiments by reducing the sensitivity and/or accuracy of dispensing location and/or dispensing volume from a fluid dispenser. For example, the absorbent element 708 may absorb and/or retain fluid splatter or peripheral fluid spray from the fluid sample dispenser, which may reduce the risk that the fluid splatter or peripheral fluid spray may migrate or deflect to contaminate adjacent structures. In some embodiments, when the dispensed fluid sample 710 is larger than what is used for a particular test, the compression of the absorbent element 708 may be configured to retain a relatively greater amount of the fluid sample 710 when compressed by the compression element 712. In some embodiments, the forces exerted by the compression member 712 may be fixed, or may be variable. A compression member 712 that exerts variable force may comprise a sensor-based feedback loop based upon sensor-feedback of the volume of the fluid sample dispensed to the transfer well, and/or a real-time feedback loop based upon the volume of the fluid sample transferred out of the transfer well.

Although the sponge element 708 depicted in FIG. 32A, comprises a single structure, in other embodiments, multiple sponge elements may be provided in a transfer well. Also, although the sponge element may fill the entire transfer well and/or extend beyond the rim of the inlet opening 702 and/or outlet opening 704 as depicted in FIG. 32A, in other embodiments, the sponge element may occupy less than the entire transfer well cavity 706, and may or may not project out of the inlet opening 702 and/or outlet opening 704. FIGS. 33A and 33B, for example, illustrates a transfer well 716 with a donut-shaped absorbent element 718 with a central opening 720. In other embodiments, the multiple openings may be provided, and/or the openings may be eccentric or comprise configurations that do not generally span or have an orientation between the inlet opening 702 and the outlet opening 704.

The absorbent element 708 may comprise any of a variety of materials in any of a variety of structural configurations (e.g. woven structure, fibrous pad, open-cell structure, etc.). The materials used may by hydrophilic, hydrophobic, lipophilic, lipophobic, etc. In some embodiments, the absorbent structure may swell with absorbed fluids, while in other embodiments, the absorbent structure may substantially maintain its pre-absorption dimensions with absorbed fluids. The absorbent materials may include but are not limited to Hydroentangled cellulose and polyester cotton fiber, polyurethane, polyethylene, latex, polyester, sponge rubber, expanded polystyrene, poly(2-hydroxyethyl methacrylate), and the like. In some embodiments, the may include or be pre-absorbed with one or more reagents, catalysts, or other substances that may initiate or facilitate the desired test reaction with the fluid sample. The absorbent element may be retained in the well cavity by a mechanical or frictional interfit, clips, sutures, and/or with adhesives. In other embodiments, the absorbent material may be kept in the well cavity by gravity.

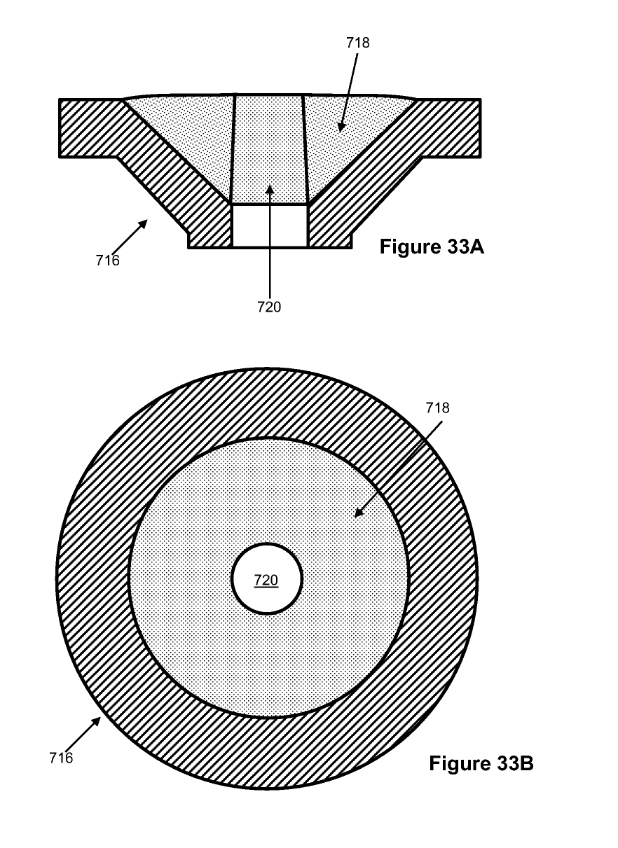

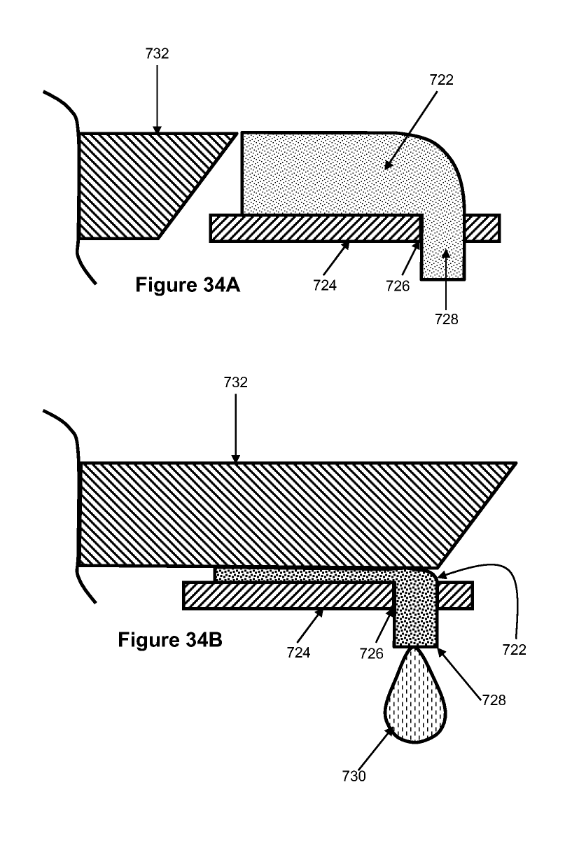

In some embodiments, the transfer structure may comprise a sponge or absorbent material that is laterally unsupported by a well, cavity or type of wall structure. In FIGS. 34A and 34B, for example, an absorbent structure 722 may be provided a base support 724 with one or more apertures 726 or fluid channels, through which an optional projection member 728 of the absorbent structure 722 may pass through.

After dispensing a fluid sample to the absorbent structure 722, the absorbent structure 722 may be compressed or squeezed to release or discharge a fluid sample 730 through the aperture 726 or other discharge region. In the particular embodiment of FIG. 34B, a single-sided compression member 732 may be used to compress the absorbent structure 722 against its base support 724. In other embodiments, however, multiple compression members may be used or a multi-faceted or circumferential compression member may be used (e.g. a funnel structure).

As noted in the particular embodiment illustrated in FIGS. 34A and 34B, the compression of the absorbent structure 722 may occur based upon side-loading by the compression member 732, but in other embodiments, the vector of the compressive forces may be different (e.g. top-loading or multi-directional loading). In some embodiments, the discharge region or site of the absorbent structure may be more centrally located or may be peripherally located or located at an end of the absorbent structure 722, as depicted in FIGS. 34A and 34B.

One or more surfaces or sections of the absorbent structure may be coated with a substance, laminate or film that may reduce the risk of peripheral discharge of the absorbed fluid sample from other sections of the absorbent structure except at the intended release site (e.g. the projection member). Also, the compression member or compression structure may be covered with a protective film or structure that may be replaced with each test, to reduce the risk that the compression structure may contaminate subsequent tests.

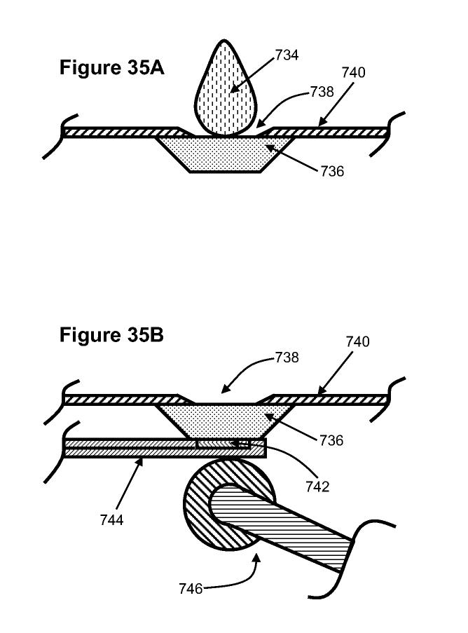

Referring to FIGS. 35A and 35B, in some embodiments, a fluid sample 734 may be dispensed to an absorbent structure 736 through an access opening 738 located on a support structure 740. The fluid sample 734 is then transferred to a test substrate 742 by using the test substrate 742 or the substrate support 744 to compress and release the fluid sample 734. The compression by the test substrate 742 and/or substrate support 744 may be facilitated by an optional compression member 746 as depicted in FIG. 35B. In this particular embodiment, the compression member 746 does not directly contact the absorbent structure 736, thereby reducing the risk of subsequent contamination by the compression member 746. Although the embodiment depicted in FIGS. 35A and 35B depicts an individual absorbent structure 736, in other embodiments, the absorbent structure 736 may comprise a continuous structure.

In FIG. 19, a ridge 352 may by used to facilitate transfer of the fluid sample 348 by reducing adhesive forces at the base 354 of the ridge 352, while isolating gravitational forces about the distal end 356 of the ridge 352 to overcome adhesive forces there. In other embodiments, the interface 362 between the base 354 of the ridge 352 and the wall 364 of the transfer well 344 may act as a capillary channel to facilitate movement of the fluid sample 344 along the ridge 352.

In some embodiments, transfer of a fluid sample from the transfer well to the test medium may occur passively. In other embodiments, however, displacement of the fluid sample out of the transfer well may be optionally facilitated by plunger, a cap or other type of pushing element, including but not limited to air or a liquid. The liquid may be generally inert and non-reactive with the fluid sample at least with respect to the particular testing performed, or may reactive to facilitate a particular type of testing.

In some embodiments, using these and other features of a transfer well may facilitate transfer of a fluid sample of a pre-determined volume within a particular tolerance level. In other embodiments, the transfer well may be configured to transfer the fluid sample dispensed from the dispensing structure within a range of volumes and with a particular tolerance range. The volume and/or mass of the sample fluid that a transfer well may transfer may depend upon the nature of chemical/biological testing to be performed on the samples. Some chemical and biological testing may use as low as 1 □liter of fluid sample per test or less, while other testing may use as much as 100 □liters or more of fluid sample per test. In other embodiments, the fluid sample used for a particular test may have a volume of about 1 □liter to about 50 □liters, and other times about 2 □liters to about 10 □liters. In some examples, the automated testing system may be configured to perform blood glucose testing using up to about 10 □liters sample volumes, lactate dehydrogenase testing using up to about 30 □liters sample volumes, and coagulation testing (e.g. Prothrombin time (PT) and partial thromboplastin time (PTT)) using up to about 100 microliters, for example. In other embodiments, however, different sample volumes may be used for the same tests, and other blood tests may also be performed. Examples of these other tests include but are not limited, to sodium, potassium, chloride, bicarbonate, creatinine, urea, albumin, total protein, alkaline phosphatase, ALT, AST, calcium, phosphorus, PO2, PCO2, pH, bilirubin (direct and total), GGT, uric acid, red blood cell count, white blood cell count, hemoglobin, hemactocrit, mean corpuscular volume, mean corpuscular hemoglobin, mean corpuscular hemoglobin concentration, red blood cell distribution width, reticulocyte count, neutrophils, lymphocytes, monocytes, eosinophils, basophils, platelet count, mean platelet volume, erythrocyte sedimentation rate, c-reactive protein, activated protein C resistance, cryoglobulins, fibrinogens, ACTH, aldosterone, ammonia, cold agglutinins, beta-HCG, magnesium, copper, iron, total iron binding capacity, transferrin, ketones, hemoglobin A1C, total cholesterol, low density lipoproteins, high density lipoproteins, triglycerides, lipoprotein A, insulin, T4 (total and free), TSH, FSH, LH, homocysteine, creatine kinase, ACTH, HIV, hepatitis BE antigen, hepatitis B surface antigen, hepatitis A antibody, hepatitis C antibody, specific gravity, RBC casts, WBC casts, and others. The diagnostic tests that may be used with the system may include tests that are commonly repeated throughout a hospital stay, as well as those that may be used less frequently.

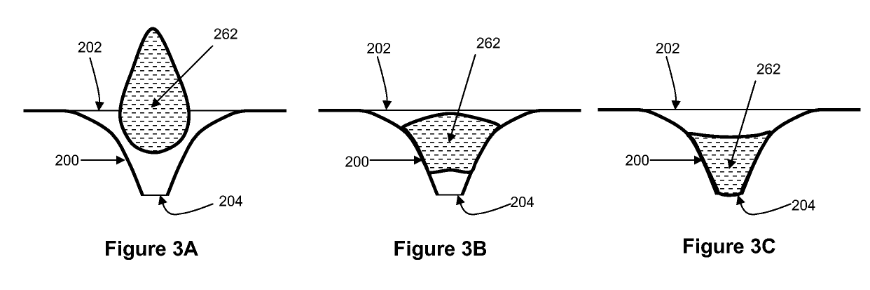

FIGS. 3A through 3C are cross-sectional views of three phases depicting a transfer well 200 of FIG. 2 being filled with a fluid sample 110. In one stage of operation depicted in FIG. 3A, a support structure (not shown), such as circular receiving disc 122 of FIG. 1, is moved to an empty transfer well 200 just below fluid droplet 262 as produced by a dispensing valve or other fluid dispensing structure (not shown). The first opening 202 of transfer well 200 is then allowed to touch the hanging fluid droplet 262, causing droplet 262 to be captured into transfer well 200. FIG. 3B shows the droplet 262 being captured in the upper portion of transfer well 200 as a result of the touch-off of transfer well 200 with the hanging fluid droplet 262. In another stage of operation, as shown in FIG. 3C, the sample fluid droplet 262 moves further down to the smaller end 236 of the funnel-shaped transfer well 200. In some embodiments, this may be due to surface tension and/or gravity. A plurality of samples of such fluid droplets can be dispensed and collected separately in the plurality of transfer wells of the disc.

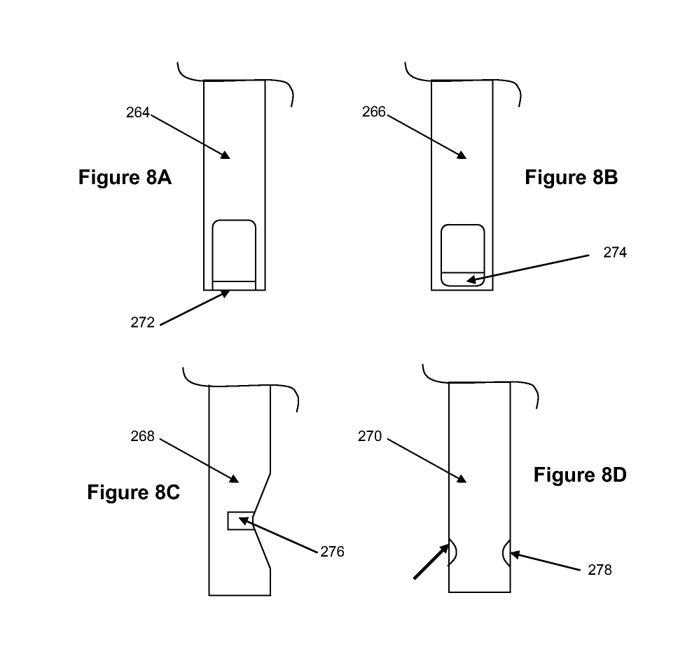

Referring back to FIG. 1, thereafter the transfer disc 122 may be moved from the sterile area 116 to a measurement area 118. At the measurement area, the outlet opening 204 of the transfer wells 108, filled with fluid sample 110, is brought in contact with a test medium 112. The test medium 112 may be provided on a customized support structure 114, or may comprise off-the-shelf test strips that may be placed into a test strip retaining structure. In some embodiments, the test medium 112 may be a test strip impregnated with one or more suitable chemical reagents for determining the concentration of an analyte in a physiological sample. Such test strips find use in the determination of a wide variety of different analyte concentrations or other characteristics, and may include but are not limited those disclosed elsewhere herein. The embodiments disclosed herein may be configured for use with any of a variety of test strips. The assessment of the test substrate or test strip may be performed using calorimetric, photometric, electrochemical or other mechanisms. Test strips that may be used include but are not limited to a variety of test strip configurations differentiated by point of reaction and/or point of sample-fill. For example, FIGS. 8A through 8D show a plurality of test-strips 264, 266, 268, and 270 with an end-fill region 272, a top-fill region 274, a single side-fill region 276, and dual side-fill regions 278, respectively.

When a test-strip or test medium is brought in contact with the lower smaller end of the transfer well, the sample fluid in the transfer well is transferred to the test-strip or test medium due to capillary action, for example. Thus, transfer wells may also be used facilitate the contained movement of fluid sample from one area to another. For example, transfer wells may also be used to provide a sterile barrier for blood testing. Referring back to FIG. 1, the dispensing device 102 would dispense into a sterile transfer well 108 of a transfer structure 104. The transfer well 108 is then removed from the dispensing area 116, reducing the risk of contaminating the dispensing system 102. The filled transfer well 108 can then be presented to non sterile test medium 112 in a measurement area 118. In some embodiments, the transfer wells 108 are configured for single use, the transfer wells 108 do not move potential contaminants from the non sterile test medium 112 back to the sterile dispensing system 102 or sterile dispensing area 116. Instead, a new sterile transfer well 108 may be used for each fluid sample 110, while the used transfer well 108 is physically removed from the sterile area to the non-sterile area, with an optional barrier in between. Embodiments utilizing a barrier are described in greater detail below.

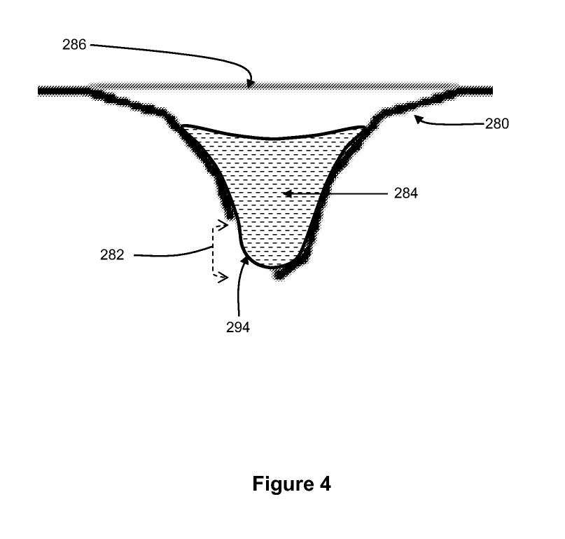

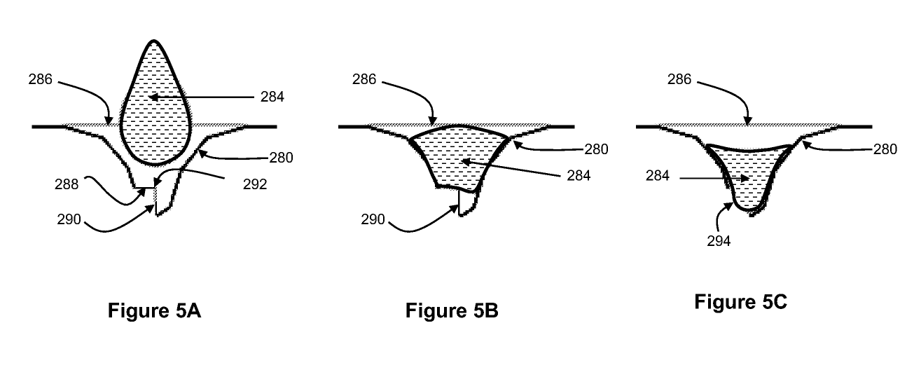

Referring now to FIG. 4, another embodiment of a transfer well 280 comprises an angled bottom opening 282. In some embodiments, a transfer well 280 with an angled bottom 282 may facilitate a particular orientation and/or configuration of the fluid sample 284 for presentation to a test medium. For example, a test strip with an end fill region as depicted in FIG. 8A may be synergistically used with an angled bottom transfer well 280. The angled bottom opening 282 of the transfer well 280 be configured to lie in a plane that is skewed in relation to the plane in which the inlet opening 286 of the transfer well 280 lies, but in the embodiment depicted in FIG. 4 and seen best in FIG. 5A, the opening 282 may be non-planar. In this particular embodiment, the edges 288 and 290 may comprise an angle 292. In some embodiments, a non-planar opening may facilitate the generation of a positive meniscus 294 in the fluid sample 284 where the outlet opening is off-axis from the inlet opening 286. FIGS. 5A through 5C illustrate a schematic cross-sectional view of transfer well 280 of FIG. 4 being filled with a fluid sample 284.

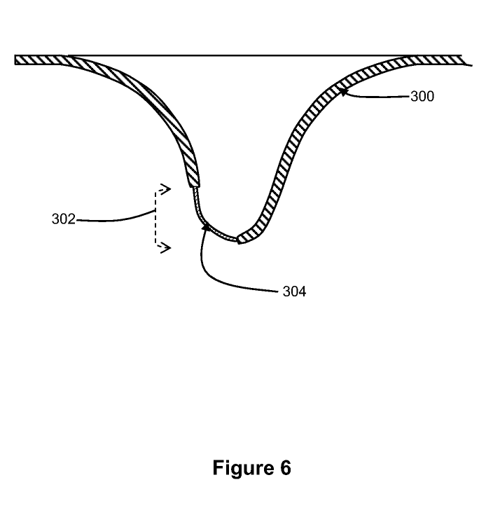

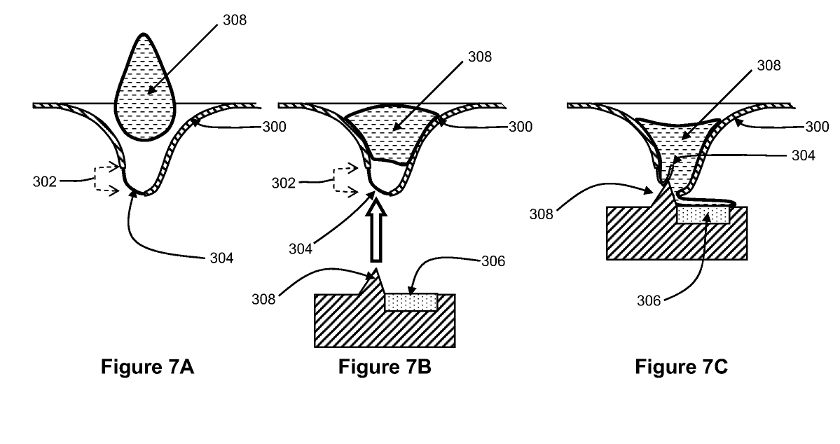

FIG. 6 is a schematic cross-sectional view of another embodiment of the transfer well 300, comprising a soft bottom outlet opening 302. In this particular embodiment, the outlet opening 302 comprises a thin material 304. In some embodiments, the material 304 has an intact surface, but in other embodiments, the material 304 may have perforations or slits formed therein. In some embodiments, one or more perforations or slits are full thickness, but in some embodiments, one or more perforations or slits may be full thickness. In some embodiments with a full thickness slit, the material 304 may be under sufficient tension such that little, if any, fluid sample leaks or otherwise presents itself through the slit. Referring now to FIGS. 7A to 7C, when presented to the test medium 306, the material 304 of the outlet opening 302 is pierced to access to fluid sample 308. In some embodiments, the test medium 306 may have any of a variety of configurations sufficient to contact or deform the material 304, but in some embodiments, the test medium 306 may further comprise a piercing member 310. The piercing member 310 may have any of a variety of projection-like configurations to penetrate the material 304. Although the embodiment depicted in FIGS. 7B and 7C comprises a sharpened piercing member 310, in other embodiments, the piercing member 310 may have a blunt tip. The piercing member 310 may have any of a variety of shapes, including but not limited to a conical or frusto-conical shape, a pyramidal shape or any other shape.

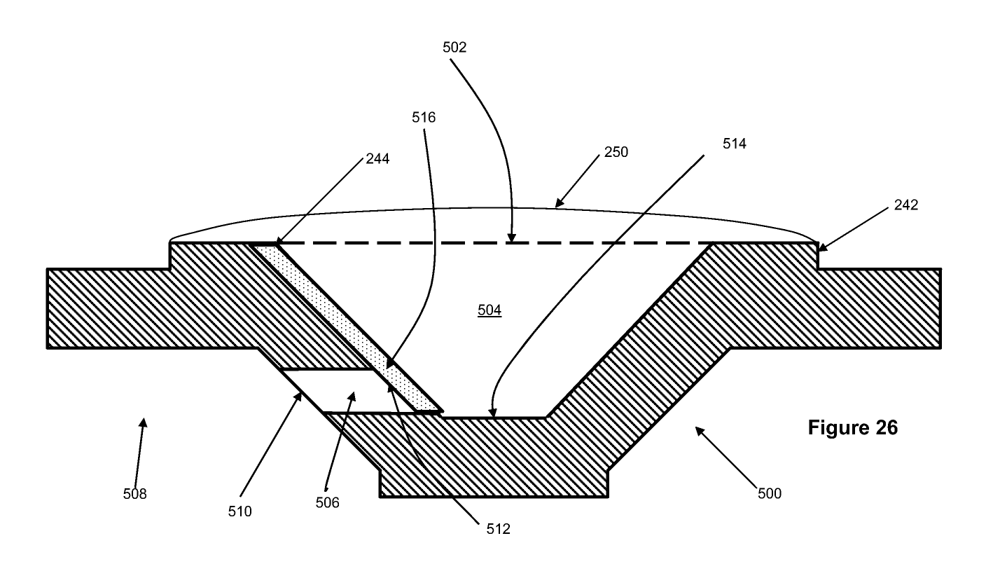

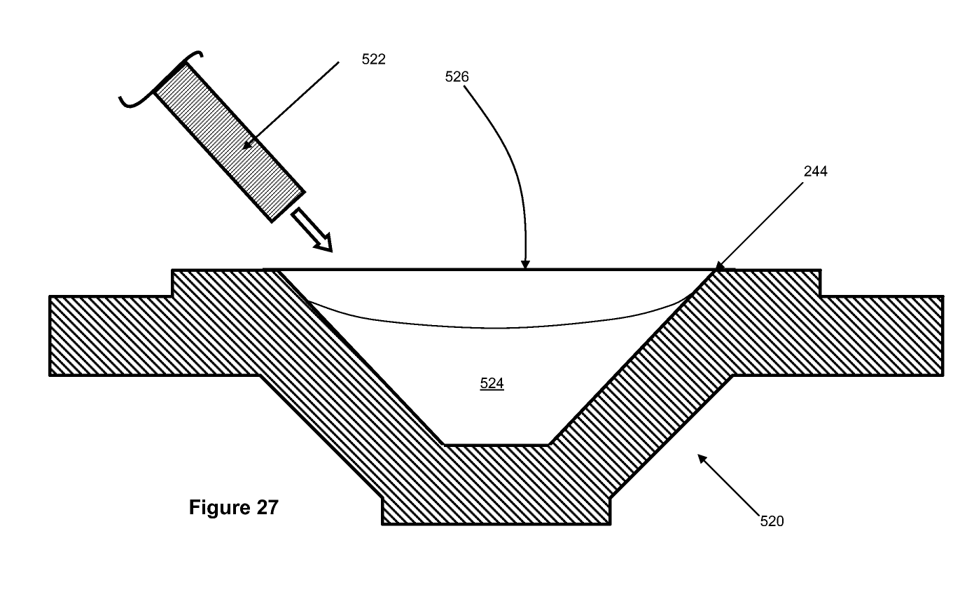

In some embodiments, the transfer well may be configured to permit the insertion of a test medium into the transfer well, instead of passing a fluid sample to the test medium through an outlet opening. In FIG. 26 for example, the transfer well 500 comprises an inlet opening 502 configured to accept a fluid sample 504 from a sample dispenser (not shown). The transfer well 500 further comprises an insertion pathway 506, which may be used to insert a test strip 508, or other test medium configuration, into the transfer well 500. In this particular embodiment, the insertion pathway 506 comprises a outer pathway opening 510 and an inner pathway opening 512, and wherein the insertion pathway 506 is generally straight and comprises a generally parallel orientation with respect to the plane of the transfer well 500. In other embodiments, the insertion pathway may have a non-linear configuration and/or a skewed or a perpendicular orientation. Also, in this particular embodiment, the inner opening 512 of insertion pathway 506 is generally located about the bottom 514 of the transfer well 500, but in other embodiments, the inner opening 512 may be located anywhere from about the inlet opening 502 to about the bottom 514. For example, the insertion pathway may be located perpendicular to the transfer well and with an inner opening at the bottom.

In some embodiments, an optional flap 516 or a membrane is provided as a barrier. This flap 516 may be located within the transfer well 500, on the outer surface of the transfer well 500, and/or within the insertion pathway 506. In some embodiments, the flap 516 is configured to resist the flow of fluid from the transfer well 500 into the insertion pathway 506, and/or to maintain the sterility of the transfer well 500 prior to insertion of the test strip 508. The flap 516 may be a flexible or a rigid member. In some instances, the flap 516 may be configured with a bias to revert to a closed position after the removal of the test strip 508, but in other embodiments remains in the open position after test strip removal. In other embodiments, a membrane structure may be provided along the insertion pathway 506 or about its openings 510 and 512, which may be pierced in order to access the transfer well 500. In some embodiments, the test strip 508 or other test medium support structure is configured with a piercing member to penetrate the membrane, but in other embodiments, a separate piercing member is used. In still other embodiments, the membrane may be configured so that no specific piercing member is needed and the end of a test strip or test medium support structure is sufficient to traverse the membrane.

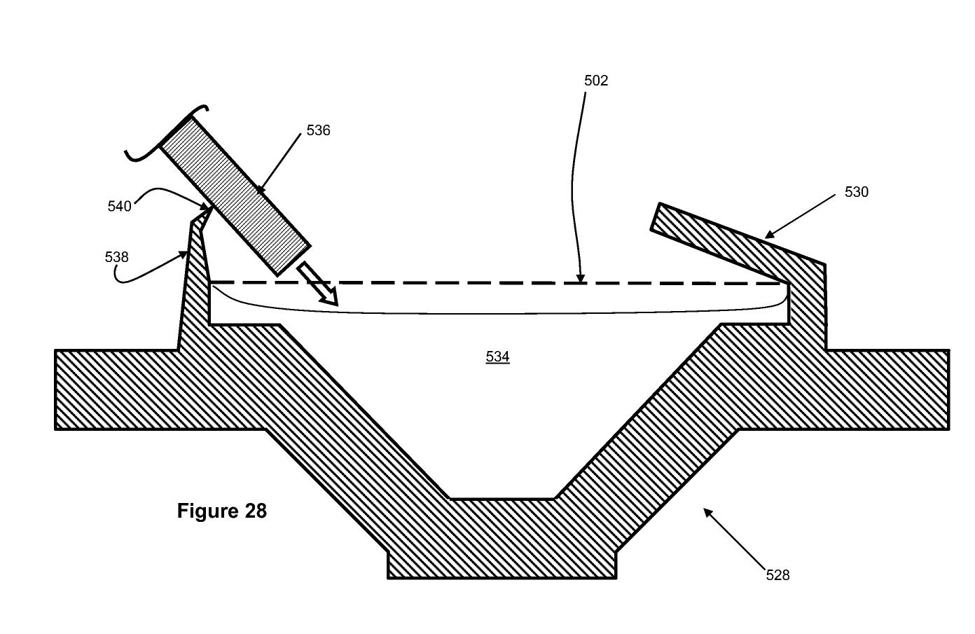

In another embodiment, the transfer well is configured to permit the insertion or contact of a test medium through the same opening which was used to receive the fluid sample. In some embodiments, the test medium or test strip may be inserted into the fluid sample, but in other embodiments, the test medium or test strip may be configured to contact the fluid sample outside of the transfer well opening. In some embodiments, a test strip with a top filling section may be used upside-down to contact the fluid sample. In other embodiments, a test strip with a bottom filling section may be used. FIG. 27 depicts one embodiment of a dip transfer well 520, wherein the test strip 522 or test medium is inserted through or contacts the fluid sample 524 about its inlet opening 526. In some embodiments, to resist significant overflow of the dip transfer well 520 by the displacement of the fluid sample 524 from the insertion of the test strip 522, the dip transfer well 520 may be underfilled relative to the inlet opening 526.

Referring to FIG. 28, in some further embodiments, the dip transfer well 528 comprises an optional cover member 530. The cover member 530 is configured to at least partially cover a portion of the inlet opening 532 of the dip transfer well 528. In some embodiments, the cover member 530 may resist contamination of adjacent transfer wells by the fluid sample 534 during interaction with the test medium 536 by containing splashes that may occur. In some embodiments, the dip transfer well 526 may optionally comprises a separation member 538 comprising an edge 540. In some embodiments where the test medium or test strip is fully saturated and portions of the fluid sample 524 remain in the dip transfer well 526, the separation member 538 may be used to separate excess fluid sample 524 that may be clinging to the test strip or test medium. In some embodiments, this may reduce the risk the contamination of the sample dispenser and/or adjacent transfer wells. In some specific embodiments, the cover member may be provided with an edge.

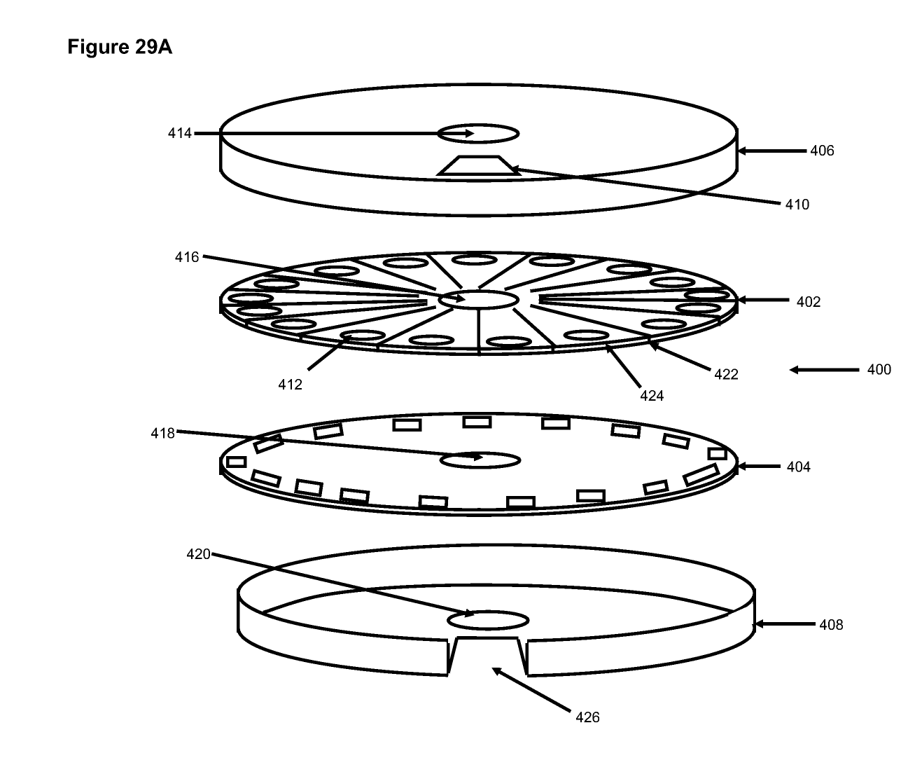

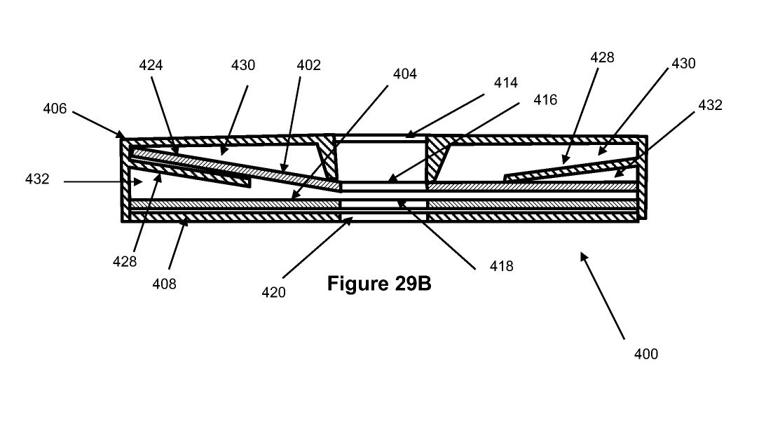

In FIG. 29A, a cartridge 400 with a transfer well plate 402 and a test medium plate 404 are provided in a housing. In this particular example, the housing comprises a dispenser housing 406 and a measurement housing 408, but in other embodiments, the housing may be integrally formed, or only comprises a dispenser housing 406, for example. Any of a variety of housing configurations are contemplated. In other embodiments, the transfer wells and the test disc may be provided in separate cartridges or modules in a test system. In other embodiments, either the transfer well plate and/or test disc may be bare, without any housing. In one specific example, a transfer well plate is provided in a housing with a dispensing aperture and a transfer aperture, and is separate from the test medium plate 404. This particular embodiment permits the sterilization of the transfer well plate and its housing, without unnecessarily sterilizing the test medium plate.

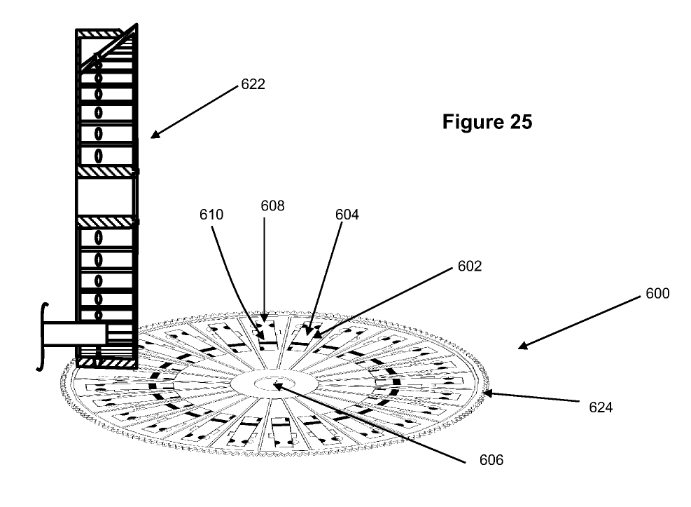

The dispenser housing 406 comprises a dispensing opening 410 which provides a pathway to dispense a fluid sample from a dispenser valve to a transfer well 412 on the transfer well plate 402, while the measurement housing 406 comprises a measurement opening 426 that provides access to the test medium plate 404. In this particular embodiment, the transfer well plate 402 and the test medium plate 404 each comprise a circular disc, but other configurations may also be used. Each component of the cartridge 400 comprises a hub opening 414, 416, 418, and 420, which may be used to access and/or manipulate the internal components or the housing components of the cartridge 400. In some embodiments, the one or more hub openings 414, 416, 418 and 420, may have a keyed configuration to permit selective manipulation of one component without substantially affecting the position of the other components. In other embodiments, one or more components of the cartridge may lack a hub opening. Although the hub openings 414, 416, 418 and 420 of the cartridge 400 depicted in FIG. 29A are centrally located, in other embodiments, one or more hub openings may be located more peripherally. In one example, the dispensing housing 408 and/or measurement housing 408 may comprise a side wall opening to access the edges of the transfer well plate 402 and/or the test medium plate 404 to cause rotation. In still other embodiments, one or more components of the cartridge may have teeth, indentations, and/or protrusions that may interface with a controller to facilitate manipulation of the one or more of the components. One example of a test medium plate or support 600 comprising edge teeth 624 that may be used to manipulate the support 600 is depicted in FIG. 25. The particular embodiment depicted in FIG. 25 will be discussed in greater detail below.

FIG. 29B is a cross-sectional schematic depiction of the assembled components 402, 404, 406, 408 of the cartridge 400 in FIG. 29A. As illustrated in FIG. 29B, the dispensing housing 404 further comprises a barrier structure 428 which separates the cartridge 400 into a first compartment 430 and a second compartment 432 in which to isolate unused transfer wells and used transfer wells, respectively. In some embodiments, the transfer well plate 402 and at least the dispenser housing 406 are sterilized such that the first compartment 430 and the tabs 424 of the transfer well plate 402 may be maintained in a sterile condition. The cartridge 400 may be assembled such that all of the tabs 424 of the transfer well plate 402 are located in the first compartment 430. As the transfer wells 412 of the transfer well plate 402 are used, they are deflected out of the sterile first compartment 430 and into the non-sterile second compartment 432, where the test medium plate 404 is located. Although the particular embodiment illustrated in FIGS. 29A and 29B depict compartments that are generally enclosed by the housing components 406 and 408, in other embodiments, for example, the dispenser housing 406 may be omitted such that the second compartment is an open compartment or region to one side of the barrier structure 428.

In the particular embodiment depicted in FIG. 29B, the transfer well plate 402 is configured so that the native or unstressed position of the tabs 424 are in the second compartment 432 while the tabs 424 are in a stressed position when located in the first compartment 430. With this particular configuration, the tabs 424 are biased so that they will deflect back into the second compartment 432 when relieved from the forces exerted by the barrier structure 428. In other embodiments, the tabs 424 may be in an unstressed position when in the first compartment 430 and in a stressed position in the second compartment 432. With this embodiment, a ramp structure or other type of deflecting structure may be provided to cause the deflection of the tab 424. In some embodiments, a motor other actuating device may be used to deflect the tab 424, either to change compartments or to facilitate transfer of a fluid sample between the sample dispense and the transfer well 412, and/or between the transfer well 412 and the test medium. In some embodiments, the ramp or deflecting structure may be integral with the barrier structure 428. In still other embodiments, the transfer well plate 402 may be configured so that its native configuration lies between the deflection positions for the first and the second compartments. In this particular embodiment, the stress is distributed between both deflection positions, and in some embodiments, this may reduce the frictional resistance of manipulating the transfer well plate 402 and/or reduce the risk of fracture or breakage of the tabs 424 by reducing the peak stresses acting on the tabs 424 that are used to deflect the tabs 424 into either compartment. Although the barrier structure 428 of the embodiment depicted in FIG. 29B is integral with a portion of the dispenser housing 406, in other embodiments, the barrier structure may comprise a separate structure, or may be integral with another component of the cartridge or module.

Depending upon the materials and/or dimensions of the transfer well plate 402 and the configuration, spacing and/or dimensions of the slits 422, in some embodiments, the tabs 424 may have a deflection range from its native position of about 0 degrees to about 90 degrees, sometimes about 0 degrees to about 45 degrees, and other times about 0 degrees to about 15 degrees or about 20 degrees. In some embodiments, the tabs 424 are configured to permit deflections in both directions or in one direction. The deflection range in one direction need not be equal to the deflection range in the other direction. In some embodiments, the deflection is elastic, such that the tab 424 may resiliently return to its native position, while in other embodiments, the deflection is plastic and the tab 424 does not return to its native position. In some embodiments, the tab 424 may break off or may separate from the rest of the transfer well plate 402 during or after use. In some embodiments, the deflection itself is sufficient to cause separation of the tab 424. In some embodiments, the deflection used to isolate unused tabs from used tabs may be sufficient or insufficient to cause separation of the tabs 424, while in further embodiments, a larger deflection is used to cause separation. In some embodiments, a cutting member or other disrupting structure is used to separate the tab 424 from the transfer well plate 402. As shown in FIG. 29B, the dispenser housing 406 is configured with an optional central spacing structure 434 to facilitate the bias provided to the transfer well plate 402. In other embodiments, a peripheral spacing structure may be provided, as well as spacing structures located on other components of the cartridge 400.

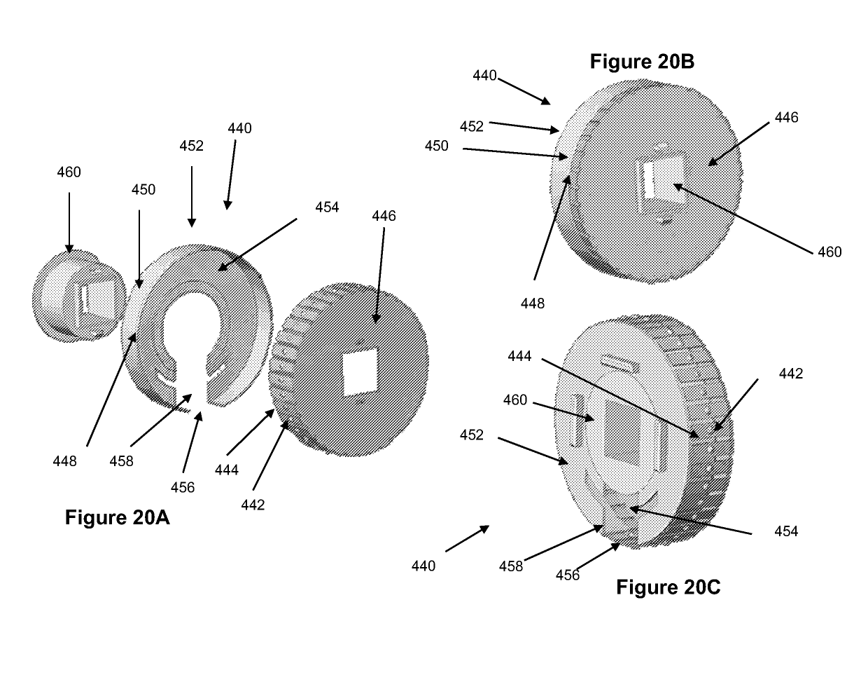

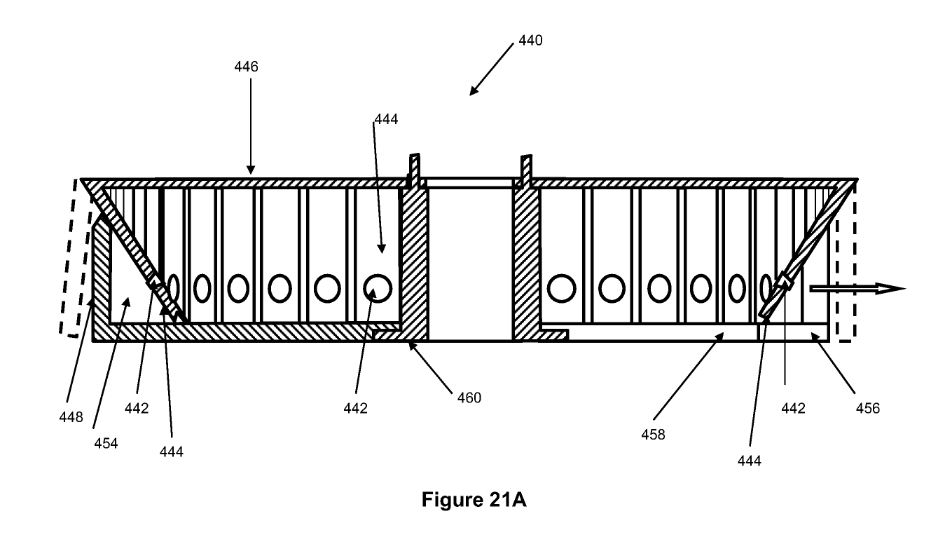

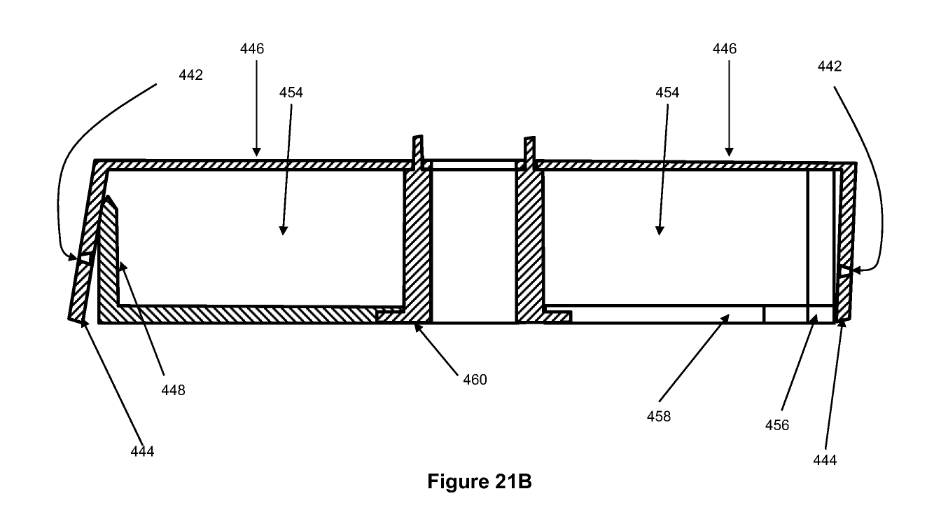

Although the barrier structure 428 depicted in FIG. 29B is configured for planar rotatable transfer well plates 402, barrier structures may also be configured for use with other the configurations and orientations of the transfer well plate and test medium plate 404. FIGS. 20A to 20C, for example, depict an embodiment of a cartridge 440 wherein the transfer wells 442 are located on tabs 444 that are angled relative to the transfer well support 446. FIGS. 21A and 21B are cross-sectional schematic depictions of the cartridge 440 from FIGS. 20A to 20C, with FIG. 21A corresponding to FIG. 20B and FIG. 21B corresponding to FIG. 20C. In this particular embodiment, the barrier structure 448 comprises a side wall 450 of a housing 452. The interior region 454 of the housing 452 may be used to provide a sterile compartment to sequester the unused tabs 444 of the transfer well support 446. When a transfer well 442 is moved into position at the transfer zone 456, the transfer well 442 may be accessed by a sample dispenser at a dispenser access opening 458. Once used, the tab 444 of the used transfer well 442 is then deflected to the other side of the barrier structure 448. FIG. 20B depicts the assembled cartridge 440 of FIG. 20A in an unused state, with all of the tabs 444 and transfer wells 446 located within the housing 452. FIG. 20C depicts the cartridge 440 of FIG. 20A after all of the tabs 444 have been rotated and deflected out of the housing 452. This is demonstrated, for example, by the lack of tabs 444 within the interior region 454 of the housing 452, as can be seen through the dispenser access opening 458. As illustrated in FIGS. 20A and 20C, this particular embodiment of a cartridge 440 includes a separate hub structure 460 which is used to couple the housing 452 to the transfer well support 446, but in some embodiments, the hub structure 460 may be integrally formed with the transfer well support 446. As noted previously, in some embodiments, the test disc or test plate may be provided separately from the transfer well disc or cartridge. In the embodiment depicted in FIGS. 20A to 20C, the cartridge 440 does not include a test medium component.

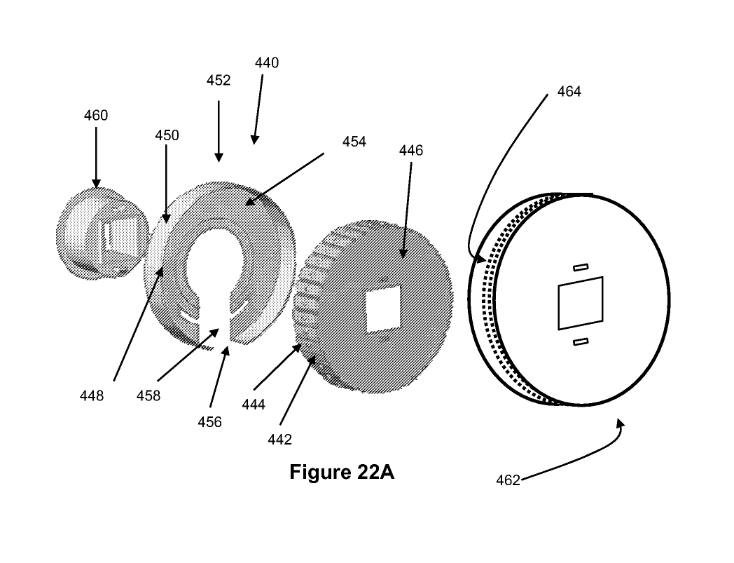

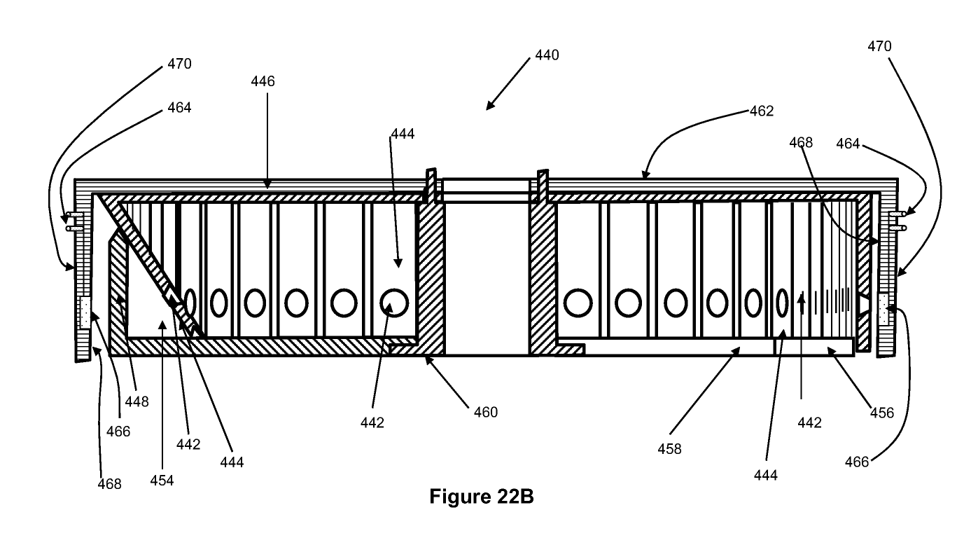

FIG. 22A depicts one embodiment wherein the module or cartridge 440 depicted in FIG. 20A, for example, further comprises a test medium support 462 comprising a plurality of test mediums (not shown). In the particular embodiment depicted in FIG. 22A, the spacing and locations of the test medium in the test medium support 462 is configured to align each of the test mediums with a transfer well 442 of the transfer well support 446. Electrodes 464 are provided on the test medium support 462 to analyze the changes in the test medium. Alternate mechanisms for assessing the test mediums are also contemplated, including but not limited to any of a variety of impedance-based processes and/or optical-based processes, such as light or laser spectroscopy. In some embodiments, the user is able to select a transfer well cartridge from a plurality of heterogeneous transfer well cartridges and coupled the selected transfer well cartridge to a test medium component selected from a plurality of heterogeneous test medium components. In some embodiments, the transfer well cartridge may be selected based upon the size or range of sizes of the transfer wells contained therein. In some embodiments, the transfer well cartridge and the test medium component may be coupled together using a snapfit, but in other embodiments, one or more fasteners may be used. In some embodiments, the complementary mechanical interfit structures between the cartridge and the test medium component may be keyed to limit the pairings between the two components. In one embodiment, for example, the keyed mechanical interfit structures may block the coupling of a cartridge with a certain type of transfer well or transfer well arrangement that would not match with a particular type of testing. In other embodiments, the test medium component may be coupled to the transfer well cartridge at the point of manufacture.

FIG. 22B is a schematic cross-sectional view through the cartridge 440 of FIG. 22A and coupled to the test medium support 462. In this particular embodiment, the test mediums 466 spaced along an inner surface 468 of the test medium support 462 with a spacing that provides an alignment to the transfer wells 442 of the transfer well support 446. Test medium 464 remains on the outside of the barrier structure 448 and therefore need not comprise tabs 444. In this particular embodiment, after reacting or interacting with the test medium 464, the test medium may be assayed or assessed using a pair of electrodes 464 found on the outer surface 470 of the test medium support 462. Also, in this particular embodiment, the cartridge 440 and the test medium support 462 may be configured so that the transfer well support 446 and the test medium support 462 are synchronously rotated by the hub structure 460, but in other embodiments, the transfer well support 446 and the test medium support 462 may be independently rotated or manipulated, or rotated as different angular amounts.

In some embodiments, the cartridge 440 is configured for use with the rotation axis of the cartridge 440 in a horizontal orientation. In other embodiments, the transfer wells and the fluid samples or other components of the automated fluid monitoring system are configured to generally utilize powered fluid sample transport mechanisms (e.g. powered droplet nozzle dispense) and/or capillary action to facilitate movement of the fluid sample. In some of these embodiments, gravitational forces are not required and therefore the axes between sample dispenser and the transfer well and/or transfer well and test medium need not be substantially vertical. In other embodiments, gravitational forces may be used to facilitate accurate transport of the fluid sample. In some of these embodiments, one or more components of the automated fluid monitoring system may have an operating range from the horizontal plane of about 0 degrees to about 45 degrees, or more, sometimes about 0 degrees to about 30 degrees, and other times about 0 degrees to about 15 degrees. In some particular embodiments, the automated fluid monitoring system may have manual and/or automatic leveling actuators to level one or more components if the usage site is not substantially level. In some embodiments, a leveling sensor may be provided to assess the horizontal level of the system. In some embodiments, the leveling sensor provides leveling information to the user and/or the control system. In some embodiments, the control system may use the leveling information to adjust the level of the system through control of the leveling actuators. In other embodiments, the leveling information provided to the user may be in the form of auditory and/or graphical or visual information. In some embodiments, the leveling information may indicate the number of degrees deviation along an X and/or Y axis of the horizontal plane. In other embodiments, the leveling information may indicate instructions for performing manual leveling of the machinery, and in some embodiments may provide a warning signal to indicate whether or not the levelness of the machinery is in an acceptable range or not.

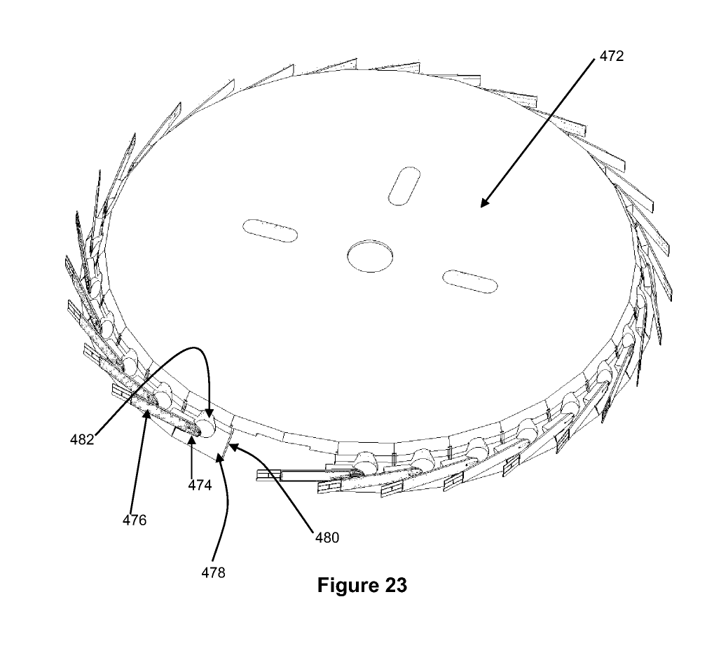

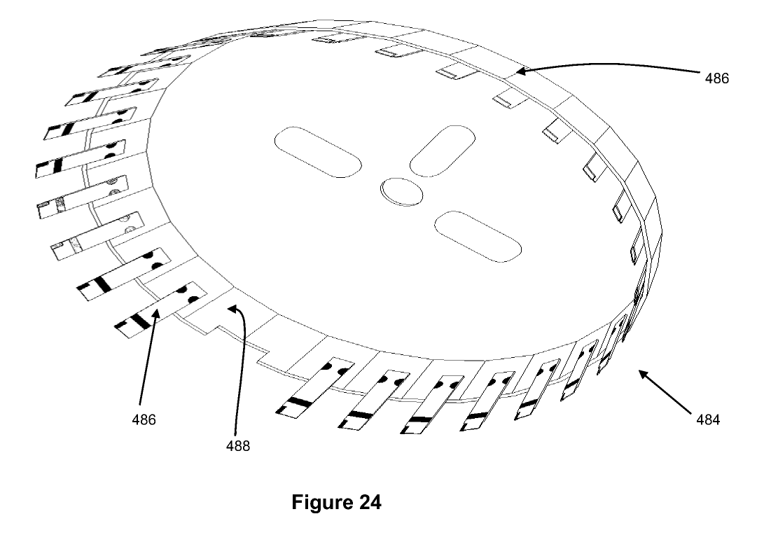

FIG. 23 depicts another embodiment of a test medium support 472 that may be used with various transfer well cartridges. In this particular embodiment, the test medium support 472 comprises one or more test strip holders 474 into which test strips 476 may be placed. In some embodiments, the test strip holders 474 may be located on the outer surface 478 of the test medium support 472, but in other embodiments, the test strip holders 474 may be located on the inner surface 480. The test strip holder 474 may comprise one or more grooves or clamping structures, for example. In some embodiments, the test strip holders 474 may form a frictional or mechanical interfit with the test strip 476 to resist separation of the test strips 476. In some embodiments, an adhesive may be provided or used to fix the test strip 476 to the test strip holder 474. In this particular embodiment, the test strip holder 474 is configured so that the test stripes 476 are oriented tangentially in generally the same plane as the test medium support 472. In other embodiments, the test strips may be angled with respect to the plane of the test medium support 472, or any other configuration, including non-tangential configurations. FIG. 24, for example, depicts another embodiment of a test medium support 484, where the test strips 486 are arranged in an angled radial configuration on an inner surface 488 of the test medium support 484, as opposed to the outer surface 486.

In the embodiment depicted in FIG. 23, the test medium support 472 further comprises transfer well apertures 482, through which fluid samples from the transfer wells (not shown) are transported. In other embodiments, transfer well apertures 482 are not provided and the transfer well may contact a test medium without requiring any intervening structures. In some embodiments, the periphery of the transfer well apertures 482 may comprise a cleaning or absorbent material. In some of these embodiments, the cleaning or absorbent material may reduce the risk of contamination of other test strips or test mediums by retaining any stray portions of a fluid sample.

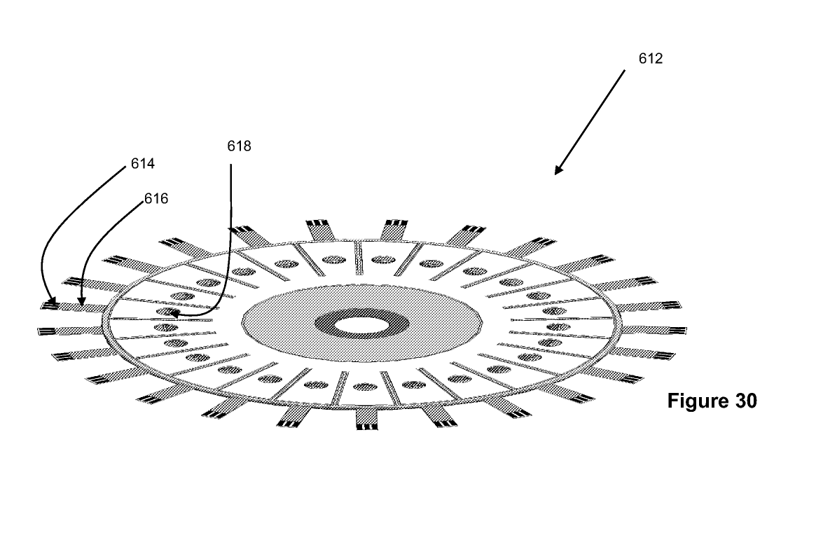

Although some of the embodiments of the test medium support described above have a generally circular arrangement on an angled surface or side wall of the test medium support, e.g. the test medium is not generally co-planar with the test medium support, in other embodiments, the test medium support may generally lack angled surfaces and/or is co-planar with the test medium support. In FIG. 25, for example, the test medium support 600 comprises a plurality of test strip recesses 602 used retain one or more test strips 604. In this particular embodiment, the test strips 604 are oriented in a radial fashion about a central hub 606 of the support 600. Although the support 600 is provided with a hub 606, the support may also be manipulated using the teeth 624 located on the edge of the support 600. In this particular embodiment, the test mediums 608 of the test strips 604 are located more peripherally while the strip electrodes 610 are located more centrally. In other embodiments, however, the orientations of the test mediums 608 and the electrodes 610 may be opposite or different from that depicted in FIG. 25. In FIG. 30, for example, the test medium support 612 is configured so that the electrodes 614 of the test strips 616 are located peripherally while the test medium 618 are located more centrally. The test medium support 612 of FIG. 30 also shows that the test strip 616 need not be fully contained within the perimeter of the test medium support 612 and may protrude outward. In this particular embodiment, the test medium support 612 further comprises an optional transfer well aperture 620. The planar test mediums, such as the test medium support 600 depicted in FIG. 25, need not be coaxial or coplanar with the transfer well plate or cartridge. In this particular embodiment, the test medium support is located at a 90 degree angle to the transfer well plate or cartridge 622, for example.

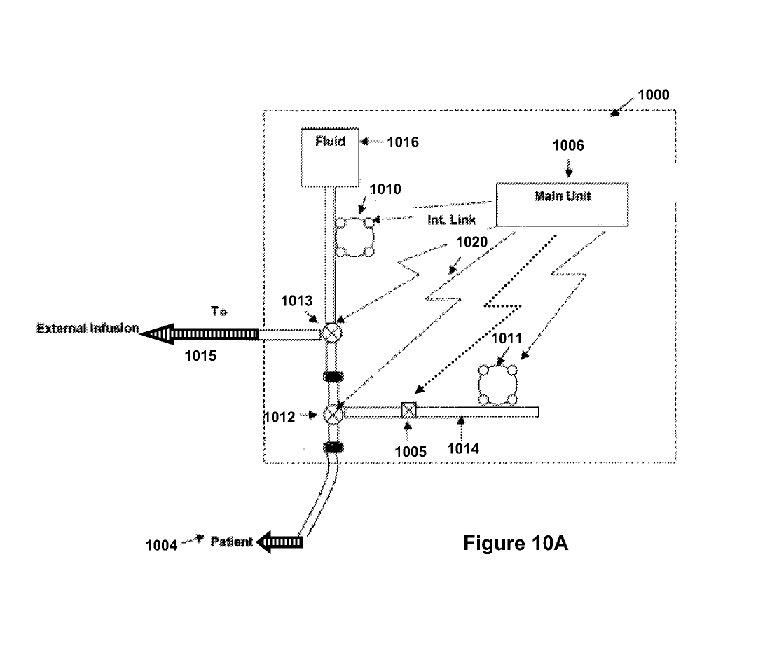

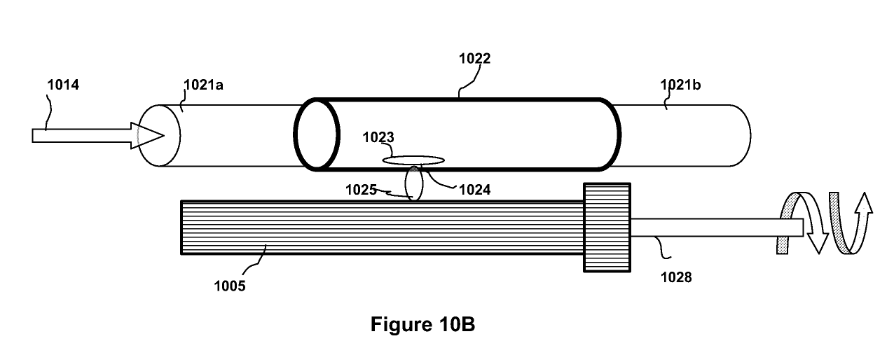

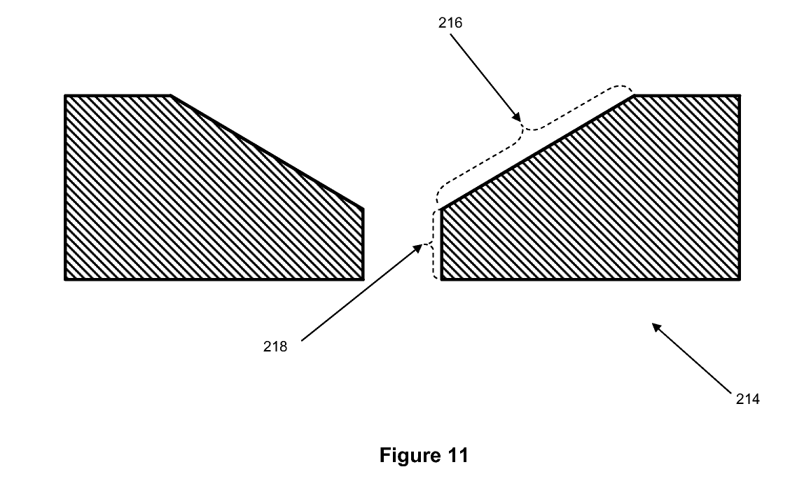

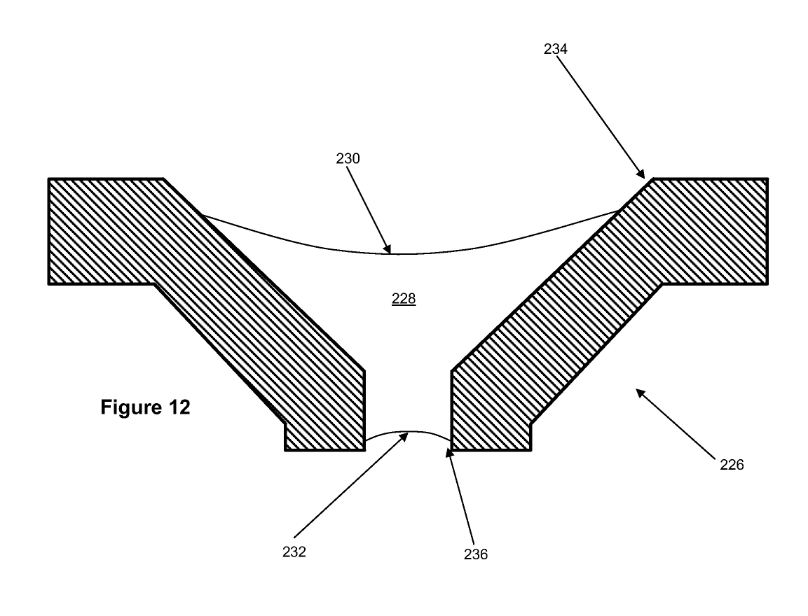

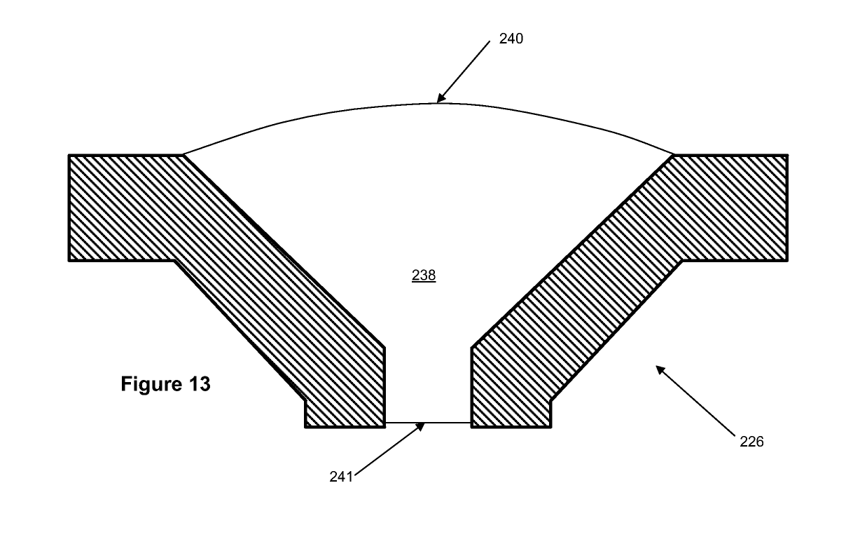

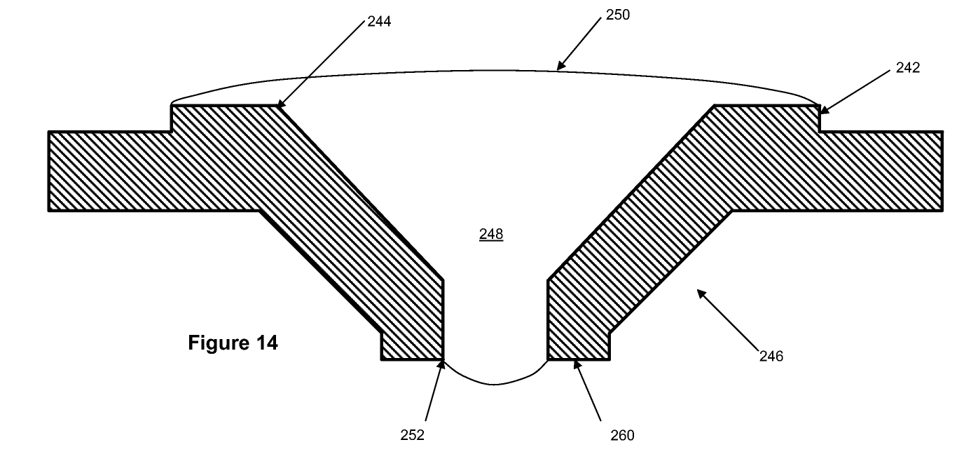

The fluid dispensing and transfer system may be deployed in combination with an automated patient fluid access device that withdraws a requisite quantity of fluid, from a patient and then transfers at least a portion of the withdrawn fluid to the fluid dispensing and transfer system. Referring to FIG. 10A, one exemplary embodiment of an automated fluid access system 1000 is shown with a fluid analysis system 1005. The automated fluid access system 1000 may be configured to communicate with a variety of sites, including but not limited to peripheral and central vascular access sites, arterial and venous vascular sites, lymphatic sites, urinary tract sites, intrapleural sites, cerebral spinal fluid sites in the spine and cranium, intraabdominal and intrapleural fluid sites, etc. In the embodiment depicted in FIG. 10A, the sample fluid is blood and therefore the foregoing description of the fluid access and sample dispensing devices, but in other embodiments, the access system 1000 and analysis system 1005 may be used with other types of fluid.

As shown in the example in FIG. 10A, the automated fluid access system 1000 may be connected to a catheter or other fluid access component (not shown) in communication with the patient 1004. A control unit 1006 may be programmed to manage control valves and other components of the access system 1000. Other components of a fluid access system may include but are not limited to pressure transducers, optical fluid detectors, in-line fluid sensors, bubble filters, RFID detectors, servo motor and motor controllers, adapters and ports for connecting to hospital and other station monitoring systems, etc. In this particular embodiment, the access system 1000 comprises a first and second pump 1010 and 1011. The pumps 1010 and 1011 may be any of a variety of suitable pumps, including but not limited to syringe pumps, peristaltic pumps, piston pumps, diaphragm pumps, etc. In some embodiments, the pumps are volumetric pumps that are configured to move a pre-specified volume of fluid, rather than a pre-specified flow rate. In some embodiments, the pumps may comprise a shared inlet/outlet port, rather than a pump with discrete inlet and outlet ports. The access system 1000 further comprises a first and second control valve 1012 and 1013. In some embodiments, the control valves are configured to control the flow along one or more fluid lines. In this particular example, the first control valve 1012 controls fluid along the fluid line 1014 which is coupled to the analysis system 1005, while the second control valve 1013 is configured to control fluid along fluid line 1015, which goes out to other components attached to the access system 1000. The analysis system 1005 may be any of a variety of compatible dispensing systems, including the dispensing and transfer system 100 depicted FIG. 1, and other dispensing and transfer systems disclosed herein, for example.

In the embodiment depicted in FIG. 1A, the first pump 1010 is configured to control the movement of a fluid sample having a pre-specified volume in the fluid delivery line from fluid container 1016, while the second pump 1011 is configured to control the flow in line 1014 used for drawing fluid samples through the dispensing device 1005. In use, the first control valve 1012 may be used to block line 1014 and keep the line 1003 to the patient 1004 open, while the second control valve 1013 may be used to permit the external infusion 1015 to flow into patient 1004 while at the same time blocking the line coming from fluid bag 1016. In other embodiments, where the multiple fluid sources are provided, the second control valve 103 may be configured to selectively access any of the multiple fluid sources. When performing automated blood sampling, the control unit 1006 may direct the second stopcock 1013 to block incoming external infusions 1015 and to open the line from fluid bag 1016 to the patient 1004. Once the external infusions 1015 are interrupted, the first pump 1010 may draw blood from the patient 1004. The blood is drawn along the tube until the remaining infusion volume and the initially diluted blood volume passes first stopcock 1012. When undiluted blood reaches the first control valve 1012, the first control valve 1012 may be repositioned to permit fluid communication between the patient 1004 and the fluid dispensing and transfer device 1005. Blood is then pumped into line 1014 via pump 1011.

When undiluted blood reaches the tube segment proximal to dispensing device 1005, blood is dispensed into a transfer well, which can then be transferred to a test medium in the form of a droplet or other volume of fluid. In addition to the access systems disclosed herein, other access systems that may be used with the dispensing systems are described in U.S. patent application Ser. Nos. 11/048,108, 11/288,031 and 11/386,078, which are hereby incorporated by reference in their entirety.

FIG. 10B depicts an exemplary embodiment of how blood, which has been withdrawn in the tubing, is transferred into the analysis system 1005. Referring to FIGS. 10A and 10B, a first tube portion 1021a receives a flow of blood 1014 that is received from the patient 1004 as a result of the pumping into line 1014 via pump 1011. The first tube portion 1021a is physically connected to a cylindrical element 1022 which comprises a dispensing port 1023. The cylindrical element 1022 is physically connected to a second tube portion 1021b that receives blood flow that is not dispensed through the dispensing port 1023.

The opening of the dispensing port 1023 may be encompassed by a cover, valve, or other structure 1024 that controls the flow of blood 1025 out of the cylindrical element 1022 and into the fluid dispensing device 1005. In some embodiments, the cover structure 1024 opens, permitting blood to flow out of the cylindrical element 1022 and into the fluid dispensing device 100.

In some embodiments, member 1028 rotates the fluid dispensing device 1005 to distribute blood to the fluid dispensing device 1005. A control unit (not shown) sends signals to a motor (not shown) causing the member 1028 to rotate.

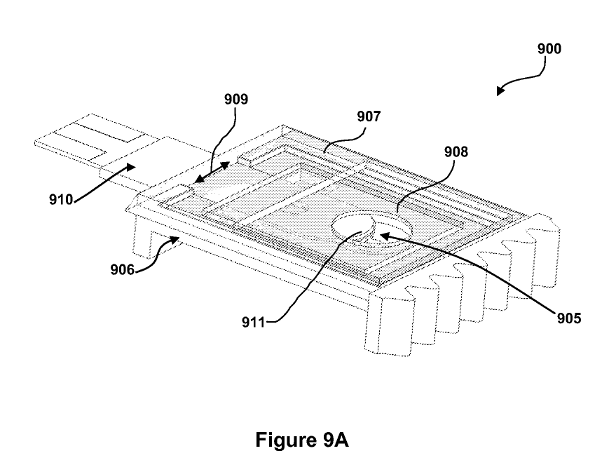

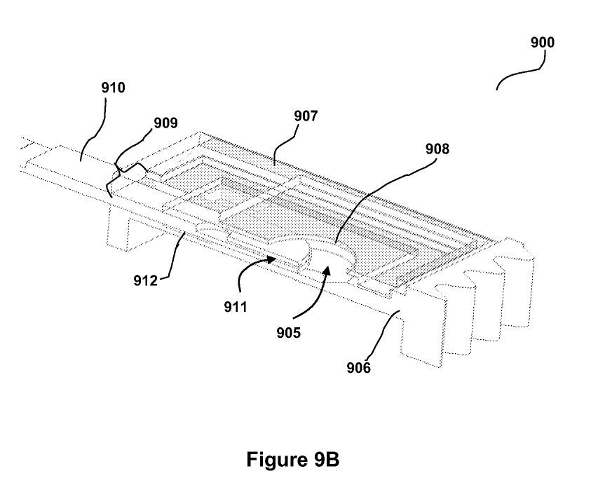

FIGS. 9A and 9B depict perspective views of another embodiment of a transfer medium. In this embodiment, the transfer medium 900 is in the form of a single-use depression or well 905 (hereinafter referred to as a ‘soak well’) located in a base support 906. A cap 907 is affixed atop the base support 906, covering the soak well 905. The cap 907 has an opening 908 that aligns with the underlying soak well 905 to facilitate capture of sample fluid in the soak well 905 through the opening 908. The cap 907 and the base support 906 also provide a side window 909 to receive a test medium 910. In one embodiment, the soak well 905 is configured as a close-bottomed half bowl shaped depression. However, in alternate embodiments, the soak well may comprise a cylindrical, funnel, rectangular, square or any other shaped depression, for example. The base 906 and the cap 907 may be made of non-toxic plastic material or biocompatible polymeric material. In one embodiment, the cap 907 is made of transparent or semi-transparent plastic to facilitate viewing of the soak well 905.

FIGS. 9A and 9B further depict a test medium 910, such as an electro-chemical glucose test strip, comprising a capillary port 911 at one end for obtaining fluid sample. During operation, the soak well 905 is brought in contact with a drop of sample fluid pending from a dispenser. The sample fluid is then transferred into the soak well 905 via opening 908. Thereafter, the capillary port end 911 of the test medium 910 is inserted into the transfer medium 900 through the side window 909 such that the capillary port 911 comes in contact with the upper surface of the fluid sample contained in the soak well 905. As a result of capillary action, an amount of the sample fluid is siphoned into the test medium 910, allowing an analyte, such as glucose in this example, in the sample fluid to be detected and/or measured. In another embodiment, the soak well 905 is configured to funnel sample fluid to the capillary port 911, thereby providing a sufficiently large area for dispensing and interfacing with the relatively very small capillary port 911.

The amount of sample fluid contained in the soak well 905 may vary. In some embodiments, the sample fluid volume exceeds the quantity of sample fluid required and obtained by the test medium 910 through the capillary port 911. In some embodiments, excess sample fluid remains in the soak well 905 may increase the likelihood of a proper test. Also, a relief 912 in the base support 906 around the test medium 910 may reduce the amount of sample fluid wicking around the test medium 910, which may allow more of the fluid to remain in the soak well 905. Additionally or alternatively, the excess sample fluid in the soak well may be wicked away to absorbent material. In some embodiments, the soak well may remove/reduce the sensitivity to under- or over dispense by acting as a ‘captured’ reservoir. In some embodiments, the soak well is configured to accept an over-delivery of volume and may utilize a capillary channel to siphon off a measured sample for analysis. The capillary channel that then samples from this well may reside within the test medium or test strip, or be captured on the periphery of the soak well and be in fluid communication to the analytical chamber/chemistry/sensor.

In some embodiments, an absorptive material provided in the soak well may also serve a ‘capturing’ function. For example, the absorptive material may be configured to absorb a greater volume of sample fluid that is needed for a test. When the test medium is inserted into the soak well, the a portion of the fluid sample in the absorptive material is transferred to the test medium, but the residual fluid sample is retained by the absorptive material and may be less likely to generate dried fluid sample particles or other potential contaminants that may interfere with proper system functioning.

In use, a fluid dispenser, including a fluid nozzle or fluid valve, may be selectively positioned over a transfer well 920 to dispense a fluid sample. In other embodiments, the fluid dispense may have a fixed dispensing location and the selected transfer well 920 of the module 924 is positioned at the dispensing location. In some embodiments, the movement pattern of the fluid dispense and/or module 924 is configured so that a used transfer well is not positioned with While in one embodiment the soak well 905 of FIGS. 9A and 9B is shown as comprised in a standalone structure, in an alternate embodiment, the soak well 905 may be part of a transfer disc, array, or a plate comprising a plurality of such soak well structure, similar to the disc 110 of FIG. 1, or other transfer support structures disclosed herein.

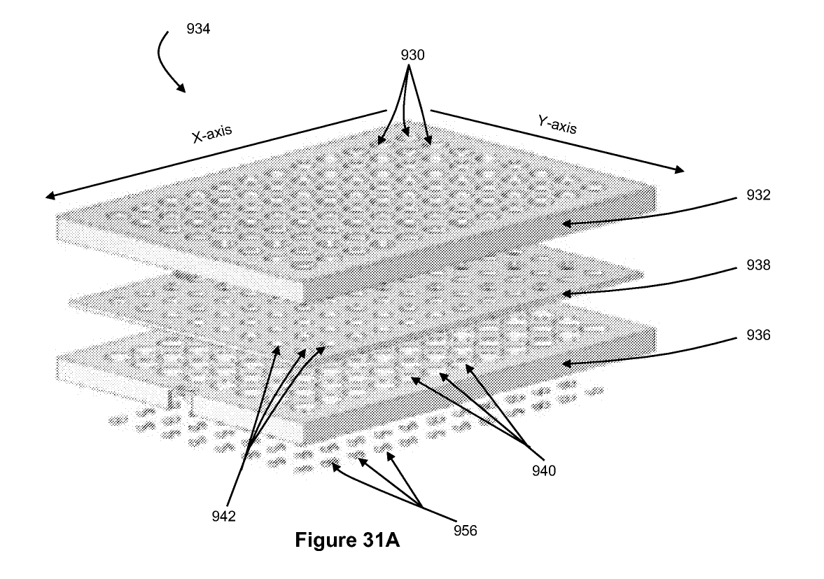

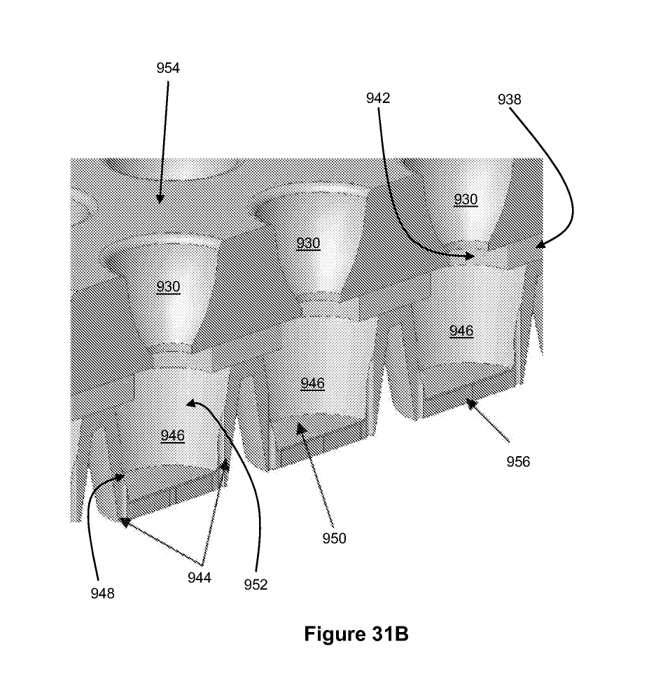

Although several embodiments described or depicted herein are directed to transfer well structures located on a disc-like rotating support structure, in other embodiments, the transfer wells may be provided on other support structures and/or support structures that utilize non-rotation movement or no movement to perform testing. In FIG. 31A, for example, the transfer wells 930 are arranged in a two-dimensional matrix on a rectangular support tray 932. In this particular embodiment, the each transfer well 930 comprises a generally uniform configuration and the spacing of the transfer wells 930 is generally uniform in both the X and Y axes. In other embodiments, the configurations of the transfer wells 930 may be heterogeneous, and/or the spacing of the transfer wells 930 may vary along the X and/or Y axes and/or between the X and Y axes. In other embodiments, the support tray 932 may comprise a non-rectangular shape, including but not limited to a circle, oval, square, triangle or other shape. In other embodiments, the support tray 932 may be also be curved or angled, or configured in any of a variety of generally three-dimensional configurations, including but not limited to a dome, cylinder, cone, cube or other type of polygonal shape. In use, the fluid dispenser and/or the support tray 932 to align the fluid dispense with the selected transfer well 930. The transfer wells 930 of the rectangular support tray may be similar to the transfer wells described in other embodiments herein, but in other embodiments, the transfer well 930 may have a different configuration.

In this particular embodiment, the rectangular support tray 932 may be a component of a cartridge or module 934 that further comprises an analyte tray 936 and a sealing barrier 938. In some embodiments, an optional sealing barrier 938 acts as a barrier between the support tray 932, which may be sterilized, and the analyte tray 936, which may not be sterilized. The sealing barrier 938 may also serve to resist contamination between analyte wells 940 of the analyte tray 936. Fluid samples in the transfer wells 930 may be transferred to the analyte wells 940 through openings 942 of the barrier 938. In other embodiments, the sealing barrier 938 may comprise one or more pores. In these embodiments, the pores permit gas or air to escape from the analyte wells 940 on the analyte tray 936. In some instances, this may reduce the risk that the transfer of a fluid sample may be blocked or hindered by the gas or air in the analyte well 940 that cannot escape or be displaced from the analyte well 940.

Referring to FIG. 31B, in other embodiments, the analyte wells 940 may be provided with one or more vents 944 to permit the escape or displacement of any gas in the analyte well 940 by a fluid sample. The analyte well 940 depicted in FIG. 31B, comprise a tapering cavity 946 with a flat bottom 948, but in other embodiments, the analyte well 930 may comprises a non-tapering cavity or comprise a tapering bottom or a non-planar bottom. In the particular embodiment of FIG. 31B, a test medium 950 is located on the bottom 948 of the well 940. In some embodiments, the test medium 950 may cover the entire bottom 948 of the well 940, but in other embodiments, may only cover a portion of the bottom 948, and/or may include a side wall 952 of the well 940. The test medium 950 may comprise a generally uniform substance or material, but in other embodiments, the test medium 950 may comprise two or more heterogeneous regions. In some embodiments, the test medium 950 may comprise a dry powder, tablet or other form factor located in the analyte well, for example. The height and/or transverse dimensions of the analyte well 940 may vary. In some embodiments, the analyte tray 936 may comprise films or layers of test medium located on a planar structure without an analyte well. In these embodiments, the analyte tray 936 may comprise separate films or layers isolated by the sealing barrier 938. In still other embodiments, the analyte tray 936 may comprise a continuous film or layer of test medium. In this embodiment, the transfer wells comprise a spacing such that the reacted portion of the continuous film or layer does not affect the portions of the continuous films or layers associated with other transfer wells.

Although not depicted in FIG. 31B, in some embodiments, the upper surface 954 may be configured with any of a variety of other structures that were described for other transfer wells herein, including but not limited to wipes and moats. In some embodiments, the bottom of the well 942 may optionally comprise an elastomeric/deformable member that can be used to pull a droplet or fluid sample into the transfer well by a vacuum. The elastomeric member can be mechanically deflected from its upper surface to deposit the droplet to the electrodes/analyte. In some embodiments, the elastomeric member may be controlled by a motor, but in other embodiments, the elastomeric member may comprise an electropolymer that may be manipulated by applying a voltage or current across the electropolymer, for example. In the embodiment depicted in FIGS. 31A and 31B, the test medium 950 may be analyzed using electrodes 956 located at the bottom 936 of the analyte well 920. In other embodiments, however, the electrodes or other sensing assemblies may be located anywhere about the analyte well 920.

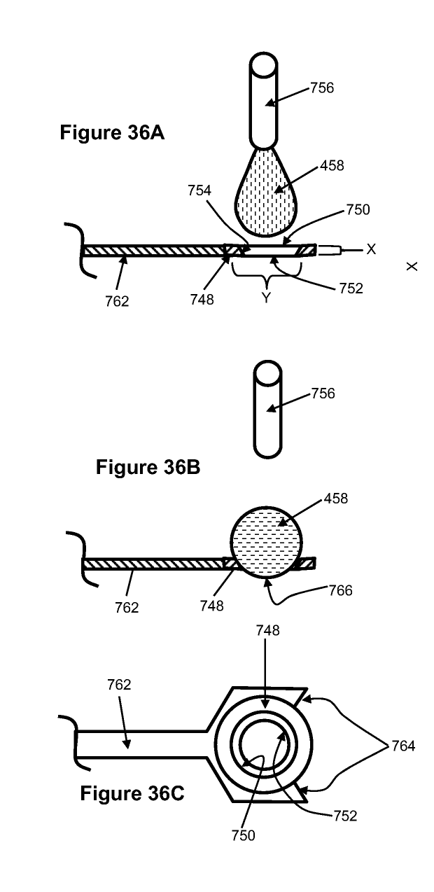

FIGS. 36A to 36C depict another embodiment of a transfer structure, comprising a ring structure 748. In this particular embodiment, the ring structure 748 has dimension X between its inlet opening 750 and outlet opening 752 that is substantially smaller than its transverse dimension Y perpendicular to dimension X. In some embodiments, dimension X is less than about 50% of dimension Y, and sometimes less than about 25% or about 15% of dimension Y. In some embodiments, the reduced dimension X of the ring structure 748 may facilitate the formation of a positive meniscus at the outlet opening 752. The ring structure 748 in FIGS. 36A and 36B comprises tapered side walls 754 from the inlet opening 750 to the outlet opening 752, but in other embodiments, the side walls may not be tapered or may taper from the outlet opening 752 to the inlet opening 750. In some embodiments, the reduced dimension X may facilitate the formation of a positive meniscus 760 in the fluid sample droplet 758.

In the particular embodiment in FIGS. 36A and 36B, the distance Z between the dispensing nozzle 756 and the ring structure 748 may remain substantially during the dispensing phase. As the fluid sample droplet 758 increases in volume, it may lengthen until it contacts the ring structure 748. Referring to FIG. 36B, in some embodiments, once the droplet 758 contacts the ring structure 748, interactive surface forces may cause the droplet 758 to reconfigure its shape, thereby facilitating its separation from the dispensing nozzle 756. In other embodiments, either the dispensing nozzle 756 and/or the ring structure 748 may moved during the dispensing phase.

In some embodiments, the ring structure 748 may be integral with a ring support 762. In other embodiments, however, the ring structure 748 and the ring support 762 may be configured to be releasably coupled to each other. In such embodiments, after the ring structure 748 is used, the ring structure 748 may be removed from the ring support 762 and a new ring structure may be reattached to the ring support 762. The coupling between the ring structure 748 and the ring support 762 may be any of a variety of interfaces, including but not limited to a mechanical interfit or a frictional interfit, for example. In some embodiments, the ring structure 748 may be separated from the ring support 762 by flicking or snapping the ring support 762, or by using an elongate member or hook to pull off the ring structure 748, for example. In the particular embodiment depicted in FIGS. 36A to 36C, the separation of the ring structure 748 may occur by reorienting the ring support 762 vertically so that the ring structure 748 is permitted to slide off the support prongs 764 of the ring support 762, illustrated in FIG. 36C.

Although embodiments have been described in relation to various examples, additional embodiments and alterations to the described embodiments are contemplated within the scope of the invention. Thus, no part of the foregoing description should be interpreted to limit the scope of the invention as set forth in the following claims. For all of the embodiments described above, the steps of the methods need not be performed sequentially.

What is claimed as new and desired to be protected by Letters Patent of the United States is:

1. A module for insertion into a fluid monitoring system, comprising:
a well support structure comprising a plurality of transfer wells, wherein each transfer well comprises a cavity comprises an inlet opening, a side wall, and an outlet opening smaller in cross-sectional area than the inlet opening;
wherein each transfer well is configured to retain a blood sample having a volume of about 1 μL to about 1000 μL and a hematocrit in the range of about 4% to about 85%.

2. The module of claim 1, wherein each transfer well is configured to form positive meniscus at the outlet opening using the blood sample.

3. The module of claim 1, wherein the well support structure is a disc structure with the plurality of transfer wells arranged in a repeating pattern.

4. The module of claim 1, wherein the repeating pattern is a circular repeating pattern.

5. The module of claim 1, further comprising a housing, in which the well support structure is contained.

6. The module of claim 5, wherein the housing comprises a fluid dispenser access opening, and a hub interface.

7. The module of claim 6, wherein the well support structure is coupled to the hub interface.

8. The module of claim 7, wherein the hub structure is a rotatable hub structure.

9. The module of claim 1, wherein each transfer well is configured to retain a blood sample having a hematocrit in the range of about 10% to about 65%.

10. The module of claim 5, wherein the housing further comprises a sterilized compartment.

11. The module of claim 10, wherein each transfer well is further configured to move from a location within the sterilized compartment to a location outside the sterilized compartment.

12. The module of claim 1, wherein each transfer well is further configured to retain a blood sample having a volume of about 1 μL to about 50 μL.

13. The module of claim 1, further comprising an absorbent structure in at least one transfer well.

14. The module of claim 13, wherein the absorbent structure extends out of the inlet opening the transfer well.

15. The module of claim 1, wherein the outlet opening is located in the side wall.

16. The module of claim 15, wherein the outlet opening comprises a first transverse dimension that is greater than a second transverse dimension that is perpendicular to the first transverse dimension.

17. The module of claim 1, wherein a separation distance between the inlet opening and the outlet opening is less than about 25% of a transverse dimension of the inlet opening.

18. The module of claim 1, wherein the separation distance between the inlet opening and the outlet opening is less than about 15% of the transverse dimension of the inlet opening.

19. The module of claim 17, wherein the outlet opening comprises a cross-sectional area that is at least about 75% of a cross-sectional area of the inlet opening.

20. The module of claim 1, wherein the cavity further comprises at least one capillary channel located on the side wall.

21. The module of claim 20, wherein at least one capillary channel comprises a radial orientation between the inlet opening and the outlet opening of the cavity.

22. The module of claim 20, wherein at least one capillary channel comprises an increased width along a direction from the inlet opening to the outlet opening of the cavity.

23. A method for performing fluid monitoring, comprising:
dispensing a fluid sample from a fluid dispenser in fluid communication with a patient;
receiving the fluid sample through an inlet opening of a transfer well;
tapering the fluid sample from the inlet opening of the transfer well to an outlet opening of the transfer well;
forming a positive meniscus of the fluid sample at an outlet opening of the transfer well; and
interacting the positive meniscus of the fluid sample with a test medium.

24. The method of claim 23, wherein the transfer well is a sterilized transfer well.

25. The method of claim 24, wherein the test medium has not been sterilized.

26. The method of claim 23, further comprising wiping the fluid dispenser with a cleaning material before dispensing the fluid sample.

27. The method of claim 23, further comprising wiping the fluid dispenser with a cleaning material after dispensing the fluid sample.

28. The method of claim 23, further comprising assessing a change in the test medium to determine a characteristic of the fluid sample.

29. The method of claim 27, further comprising advancing a new transfer well toward the fluid dispenser.

30. The method of claim 29, wherein wiping the fluid dispenser occurs while advancing the new transfer well toward the fluid dispenser.

31. The method of claim 23, further comprising moving a new test medium while advancing the new transfer well.

32. A system for manipulating a fluid sample, comprising:
a fluid access device configured to be secured to a patient;
a fluid sample dispenser;
a plurality of fluid test media; and
a plurality of wells comprising an inlet opening and an outlet opening, wherein the inlet opening is configured to accept a fluid sample from the fluid sample dispenser and the wherein the outlet opening is configured to deliver the fluid sample to a fluid test medium and wherein the outlet opening has a smaller cross-sectional area than the inlet opening.

33. The system of claim 32, further comprising a fluid pump.

34. The system of claim 32, further comprising at least one fluid source.

35. The system of claim 32, further comprising at least two fluid sources and a distribution valve configured to selectively communicate with at least two fluid sources.

36. A system for manipulating a fluid sample, comprising:
a fluid pathway between a patient and a test medium;
a sterilized fluid dispenser located within the fluid pathway;
at least one sterilized tapered transfer well within the fluid pathway;
at least one bridgeable gap within the fluid pathway between the sterilized fluid dispenser and at least one sterilized transfer well; and
a plurality of single-use test substrates.

37. The system of claim 36, wherein the plurality of single-use test substrates comprise irreversible reaction reagents.

38. A system for manipulating a fluid sample, comprising:
a fluid access device configured to be secured to a patient;
a fluid sample dispenser;
a plurality of test substrates; and
a plurality of retaining structures comprising an opening, wherein the opening is configured to accept a fluid sample from the fluid sample dispenser.

39. The system of claim 38, further comprising a test substrate advancement mechanism configured to insert at least one of the plurality of test media into at least one of the retaining structures.

40. The system of claim 39, wherein the plurality of retaining structures further comprises an insertion opening, and wherein the test media advancement mechanism is configured to insert at least one of the plurality of test media into the insertion opening.

41. The system of claim 38, wherein the retaining structure is an absorbent structure.

42. The system of claim 41, wherein the retaining structure is selected from a group consisting of a foam, fibrous structure and a woven structure.

43. The system of claim 41, wherein the retaining structure further comprises a well cavity in which the absorbent structure is located.

44. The system of claim 38, wherein the plurality of test substrates comprise a plurality of irreversible reaction reagents.

45. The system of claim 41, further comprising a compression structure.

46. The system of claim 41, wherein the compression structure is configured to press a test substrate of the plurality of test substrates against a retaining structure of the plurality of retaining structures.

47. A method for performing fluid monitoring, comprising:
dispensing a fluid sample from a fluid dispenser in fluid communication with a patient;
receiving the fluid sample through an opening of a retaining structure;
contacting a test substrate to the retaining structure;
transferring at least a portion of the fluid sample from the retaining structure to the test substrate;
positioning the test substrate at an analysis site;
analyzing the test substrate with a sensor assembly at an analysis site; and
removing the test substrate from the analysis site.

48. The method of claim 47, further comprising absorbing the fluid sample with the retaining structure.

49. The method of claim 47, further comprising irreversibly reacting the fluid sample with the test substrate.

50. The method of claim 47, further comprising inserting the test substrate through the opening of the retaining structure.

51. The method of claim 50, wherein inserting the test substrate through the opening of the retaining structure is performed before transferring at least a portion of the fluid sample from the retaining structure to the test substrate.

52. The method of claim 47, further comprising inserting the test substrate through an insertion opening of the retaining structure, wherein the insertion opening is different than the opening of a retaining structure that receives the fluid sample.

53. The method of claim 47, further comprising compressing the retaining structure using the test substrate.

54. The method of claim 53, further comprising applying force against the test substrate using a compression member.

55. The method of claim 47, wherein removing the test substrate from the analysis site comprises pivoting the test substrate away from the analysis site.
